(19) United States
(12) Patent Application Publication
(10) Pub. No.: US 2023/0265476 A1
(43) Pub. Date: Aug. 24, 2023

(54) A METHOD AND MODULAR APPARATUS FOR THE SYNTHESIS OF RNA-BASED THERAPEUTICS

(71) Applicant: Charalampos Makatsoris, Bedfordshire (GB)

(72) Inventor: Charalampos Makatsoris, Bedfordshire (GB)

(21) Appl. No.: 17/924,965

(22) PCT Filed: May 12, 2021
(86) PCT No.: PCT/GB2021/051132

§ 371 Date: Nov. 11, 2022

(30) Foreign Application Priority Data
May 12, 2020 (GB) 2006972.0
May 12, 2020 (GB) 2006975.3

(51) Int. Cl. C12P 19/34; C12Q 1/6806; B01L 3/00

(52) U.S. Cl. CPC C12P 19/34; B01L 3/502753; C12Q 1/6806; B01L 2300/044; B01L 2300/0861

(57) ABSTRACT

A method of RNA synthesis via fluid flow apparatus. The method may involve introducing into a first fluid flow module, via a plurality of inlet ports, a plurality of reactants comprising: at least one nucleoside triphosphate (NTP), a reaction buffer, and DNA, a DNA based compound or a DNA based mixture; allowing at least some of the reactants to react within a reaction channel or well within the first module of the flow system; retaining or recirculating the DNA at the first reactor module and allowing reaction products of the reactants to flow into a first fluidic filtration module; and filtering the reaction products within the first filtration module.

### FIELD OF INVENTION

The present invention relates to a method of RNA synthesis and in particular, although not exclusively, in a scalable and modular fluid flow system and method for the continuous, automated, or semi-automated RNA synthesis.

### BACKGROUND ART

Flow reactors, alternatively termed continuous flow reactors, provide for a continuous flow of materials or reactants through a network of conduits connected to form a passage of fluid. Fluid communication is enabled through a variety of configurations of the conduit network by enabling or disabling fluid passage with ports and valves when assembling the mixing, reaction and filtration modules of the flow system, to control the conditions of the synthesis, purification and formulation of the RNA or nucleic acid therapeutic for the manufacture of product in continuous flow format. A computer system operating a purposely constructed software code enables the directed control of the entire set of process parameters within the flow system. These include mixing conditions, temperature, pH, reagent concentrations, monitoring, residence times, purity profile, yield etc. Example prior art flow reactors are typically formed as an assembly of individual modules that are connected face-to-face to form a unitary block through which a fluid is directed. Example flow reactors are described in WO 2013/050764.

Flow technologies offer more sustainable, flexible and effective production of pharmaceutical manufacture. Combined with micro-technologies and precision engineering flow systems can be constructed and configured as micro-factories that integrate a plurality of unit operations such as mixing, reaction synthesis, extraction, separation, filtration and purification. Furthermore, the small volume to surface area ratio that is typical in flow mixing and reaction systems allow precise control of the process conditions with respect to heat and mass transfer, thereby accelerating the process and increasing productivity at small device footprints. Integrated flow systems combining these unit operations can be considered as micro-factories or downsized production systems that decrease the use factory floor space, reduce energy consumption and improve resource utilisation. Accordingly, it is possible to achieve a sustainable growth with better environmental impact, cost effectiveness and flexibility compared to current manufacturing systems used typically for the production of substances as prophylactic vaccines currently.

There are several protocols in molecular biology and biotechnology that increasingly use Lab-on-chip (LOC) devices. Such LOC devices use micro-manufacturing techniques to miniaturize and integrate laboratory analysis assays and perform small scale synthesis or filtration into a small chip. These small devices consume less material and produce less waste reducing the cost and also allow faster response time. A variety of steps of given protocols can be integrated into a LOC device by directing fluid flow through, micromixers, microchannels, filters, and allow cell sorting, mixing and enable reactions to take place for the synthesis of DNA or RNA for example. It is important that LOC devices are formed from reaction-inert materials and are preferably transparent, in order to enable visual inspection of the reaction in real-time in the chip. Also, a device that is transparent, enables the possibility for spectroscopic analysis and investigations. A preferred material used for the rapid prototyping of such applications is PDMS (Polydimethylsiloxane).

In 2019, the World Health Organization identified ten worldwide threats to the global health. Eight of these ten threats are dealing with pandemics and accessibility to vaccines especially in LMIC (low- and medium-income countries) where pandemics such as Ebola or Dengue have/are impacting population.

Therefore, there is an urgent need for apparatus and methods suitable for use in the manufacturing of vaccines and vaccine pre-cursors that can meet the demands of large quantities, low cost and rapid bulk synthesis. In particular there is a need for a chemical compound manufacturing platform that can be used to meet urgent vaccine demand i.e., within a couple of days, is easy to store and deploy around the world, and is highly productive and cost effective to run.

### SUMMARY OF THE INVENTION

Accordingly, it is an objective of the present invention to provide a modular manufacturing platform capable of scalable synthesis and/or filtering of chemical compounds and biological and non-biological molecules including in particular RNA-based vaccines or other nucleic acid therapies. A particular aim of the present invention is to provide a configurable and integrated micro-factory flow system for the preparation of nucleic acid based vaccines manufacture.

The present disclosure provides a modular and integrated device that is inspired from these methodologies and laboratory protocols that includes a configurable and highly scalable method for the manufacture of RNA and nucleic acid substances rapidly and ultimately at the point of use, within for example a hospital setting.

In particular, the present system comprises a combination of a flow reactor unit, a ‘continuous’ filtration unit and a mixing unit. These units may be integrated in any sequence appropriate to a target RNA, Nucleic Acid therapeutic or prophylactic vaccine. The present integrated flow system may comprise several optional analytical probes including a fibre-optic probe, detector and light source for UV-Vis absorbance or fluorescence spectroscopy for the in-situ and in-line control of the synthesis or manufacture of the target substance within a continuous flow process.

A specific objective is to provide a microfluidic-based apparatus and method integrating modules including a fluid flow bioreactor module and fluid flow filtration module interconnected via respective conduits/channels to create a continuous fluid flow pathway that may be operated continuously and may be automated using electronic and/or software control utilities involving the use of membranes, component actuators such as pumps, valves, gates, delivery ports, outlet ports, syringe pumps, sensors etc. as will be appreciated.

It is a specific objective to provide a fluid flow system for the reaction synthesis of selected chemical components such as biological and non-biological molecules via a process that may be continuous or non-continuous and that may be automated or semi-automated. It is a further specific objective to provide a modular system in which individual modular units may be interconnected depending upon the synthetic pathway. Such units may comprise ports, valves and suitable connections to enable the fluid communication and interconnection between respective modules. It is a further specific objective to provide a reaction system for a fluid configurable to measure and be responsive to characteristics of the system such as pressure, temperature, pH, a volume or ratio of one or more selected chemical components of the fluid, a flow rate of the fluid and a reaction status of the fluid.

It is a further specific objective to provide a fluid flow filtration apparatus and method in which chemical components of a fluid may be separated within an automated or semi-automated fluidic flow system. It is an objective to provide a filtration system that may be operated continuously via suitable electronic control and fluid delivery, recirculation and/or driving via fluid pressure. It is a further specific objective to provide a fluid flow system having one or a plurality of reactor modules and filtration modules interconnected to form a network.

It is a further objective to provide a method and apparatus for the synthesis of RNA. It is a further specific objective to provide apparatus and method for the synthesis of RNA from DNA. It is a yet further objective to provide a form of synthetic RNA that may be used for the preparation of a vaccine.

It is one objective of the present invention to provide a modular manufacturing platform capable of synthesis and/or filtering of chemical compounds and biological and non-biological molecules including in particular RNA and subsequently vaccines. It is a specific objective to provide apparatus and a system configurable as a micro factory for biological molecule synthesis to enable downstream vaccine manufacturing.

It is a specific objective to provide apparatus and method forming a fluid flow integrated system comprising component modular parts including fluid flow bioreactor modules and fluid flow filtration modules interconnected via respective conduits/channels to create a fluid flow pathway that may be operated continuously and may be automated using electronic and/or software control utilities.

According to a first aspect of the present invention there is provided a method of RNA synthesis comprising: introducing into a first fluid flow module, via a plurality of inlet ports, a plurality of reactants comprising: at least one nucleoside triphosphate (NTP), a reaction buffer, and DNA, a DNA based compound or a DNA based mixture; allowing at least some of the reactants to react within a reaction channel or well within the first module of the flow system; retaining or recirculating the DNA at the first reactor module and allowing reaction products of the reactants to flow into a first fluidic filtration module; and filtering the reaction products within the first filtration module.

Optionally, the at least one nucleoside triphosphate (NTP) is a solution of at least one nucleoside triphosphate (NTP).

Optionally, the at least one nucleoside triphosphate (NTP) comprises any one or a combination of adenosine triphosphate (ATP); cytidine triphosphate (CTP); guanosine triphosphate (GTP); uridine-triphosphate (UTP).

Optionally, the DNA is plasmid DNA. Optionally, the plurality of reactants further comprise any one or a combination of an enzyme mix, a salt solution, RNA polymerase.

Optionally, the method comprises recirculating at least some of the plurality of reactants from an outlet of the first filtration module to an inlet region of the first reactor module. Optionally, the method comprises delivering at least some of the filtered reaction products from the first filtration module into a second fluid flow reactor module in combination with inputting a capping enzyme into the second reactor module. Optionally, the method comprises delivering the fluid output from the second reactor module to a second fluid flow filtration module. Optionally, the method comprises recirculating any unreacted NTP to an inlet region of the first reactor module and recirculating any unreacted capping enzyme to the inlet region of the second reactor module.

Optionally, the salt solution or buffer comprises MgClz. Optionally, the RNA polymerase comprises T7 polymerase.

According to a further aspect of the present invention there is provided RNA or an RNA based compound prepared by the method as claimed herein.

According to a further aspect of the present invention there is provided use of RNA or an RNA based compound prepared by the method as claimed herein in the preparation of a vaccine.

According to a further aspect of the present invention there is provided fluid flow filtration apparatus comprising: a first elongate fluid flow channel having an inlet; a second elongate fluid flow channel having an outlet; a permeable membrane positioned to partition the first channel from the second channel along the respective lengths of the first and second channels such that a permeate may pass from the fluid within the first channel via the membrane into the second channel along the lengths of the first and second channels.

Optionally, a majority of the length of the first channel is positioned adjacent a majority of the length of the second channel via the membrane.

Optionally, a pore size of the membrane is in a range 100 to 1000 kDa; 100 to 800 kDa; 200 to 800 kDa or 200 to 600 kDa; 300 kDa to 10 MDa.

Optionally, the apparatus comprises a first plate in which the first channel is formed and a second plate in which the second channel if formed, the membrane sandwiched between respective opposed faces of the first and second plates. Optionally, the first and second channels each comprise a series of straight sections and bent sections. Optionally, the first and second channels comprise respective serpentine profiles in their lengthwise directions. Optionally, the first and second channels are open along their lengths and positioned in direct contact with the membrane that, in part, defines a lengthwise wall or face of the first and second channels. Optionally, a pore size of the membrane is less than an average molecular size of an RNA molecule.

According to a further aspect of the present invention there is provided a fluid flow system for the processing of a fluid comprising: a first reactor module having a reaction flow channel or well, at least one inlet and at least one outlet; a first filtration module having a fluidic filtration region, at least one inlet provided in fluid communication with the outlet of the first reactor module and at least one outlet; wherein the first filtration module comprises the fluid flow filtration apparatus as claimed herein.

Optionally, the system comprises a second reactor module having a reaction flow channel or well, at least one inlet and an outlet, said inlet provided in fluid communication with the first filtration module.

Optionally, the system comprises a second filtration module having a fluid filtration region, at least one inlet and outlet, said inlet provided in fluid communication with the outlet of the second reactor module.

Optionally, the system comprises a fluid injection port provided in fluid communication with an inlet region of the first filtration module. Optionally, the system comprises a first recirculation conduit extending between a region of the outlet of the first reactor module and the at least one inlet of the first reactor module. Optionally, the system comprises a second recirculation conduit extending between a region of the outlet of the first filtration module and the outlet of the first reactor module. Optionally, the system comprises a third recirculation conduit extending between a region of the outlet of the second filtration module and the inlet of the first reaction module.

According to a further aspect of the present invention there is provided a method of filtering a fluid using fluid flow filtration apparatus comprising: driving a fluid through a first elongate fluid flow channel from an inlet; forcing a permeate component of the fluid through a membrane extending along the first channel and into a second elongate fluid flow channel; and retaining a retentate component of the fluid within the first channel; wherein the membrane is positioned to partition the first channel from the second channel along their respective lengths such that the permeate may pass from the first channel via the membrane into the second channel along the respective lengths of the first and second channels.

According to a further aspect of the present invention there is provided a flow system comprising: at least one reactor module having a reaction fluid flow channel or well, at least one fluid inlet and at least one fluid outlet; at least one flow driver to drive a flow of a fluid through the channel or well; a first sensor to measure any one or a combination of a pressure, a temperature or a pH of the fluid within the system; a second sensor to measure any one or a combination of a pressure, a temperature, a pH of the fluid within the system; a reaction status monitoring device to monitor a characteristic of a fluid within the system indicative of a status of reaction of at least two chemical components of the fluid within the system; a control unit to receive data from the at least one or a combination of the first sensor, the second sensor and the reaction status monitoring device and to control at least one characteristic of the fluid within the system.

Optionally, the characteristic of the system is any one or a combination of: a pressure of the fluid within the system; a temperature of the fluid within the system; a pH of the fluid within the system; a volume or ratio of one or more chemical components of the fluid within the system; a flow rate of the fluid within the system. Optionally, the step of driving the fluid comprises pressurizing the fluid within the first channel. Optionally, the control unit comprises a CPU, a PCB, a PLC, a PC, a processor chip, a handheld electronic device. Optionally, the additional sensors comprise any one or a combination of temperature, pH, pressure, flow rate, flow volume or spectroscopic sensors.

According to a further aspect of the present invention there is provided a method of processing a fluid using fluid flow apparatus comprising: introducing at least one fluid into a reactor module via at least one inlet; driving the fluid to flow through or within a reaction flow channel or well using at least one flow driver and outputting the fluid at an outlet; measuring at least one or a combination of pressure, temperature or pH of the fluid within the fluid flow apparatus; monitoring a reaction status of chemical components within the fluid within the fluid flow apparatus; and controlling at least one characteristic of the fluid within the fluid flow apparatus using a control unit in response to at least one or a combination of the measuring of the pressure, pH, temperature and/or reaction status of the chemical components of the fluid.

Optionally, the system comprises a plurality of reactor modules and filtration modules coupled together in fluid communication. Optionally, the flow driver is at least one pump and optionally a syringe pump. Optionally, the fluid analysis sensor comprises a UV-Vis spectroscope for absorbance or fluorescence analysis in the range 190 nm to 1000 nm.

Optionally, the method of RNA synthesis comprises recirculating any unreacted NTP to an inlet region of the first reactor module and recirculating any unreacted capping enzyme to the inlet region of the second reactor module.

According to a further aspect of the present invention there is provided RNA prepared by the method as claimed herein. According to a further aspect of the present invention there is provided use of RNA prepared by the method of any preceding claim in the preparation of a vaccine.

According to a further aspect of the present invention there is provided fluid flow apparatus for processing a fluid comprising: a first reactor module having a reaction flow channel or well, at least one inlet and at least one outlet; a first filtration module having a fluidic filtration region, at least one inlet and at least one outlet, said inlet provided in fluid communication with the outlet of the first reactor module.

Optionally, the apparatus comprises a second filtration module having a fluidic filtration region, at least one inlet and an outlet, said inlet provided in fluid communication with the outlet of the second reactor module.

Optionally, the apparatus comprises a fluid injection port provided in fluid communication with an inlet region of the second reactor module. Optionally, the apparatus comprises a first recirculation conduit extending between a region of the outlet of the first reactor module and a region of the at least one inlet of the first reactor module.

Optionally, the apparatus comprises a third recirculation conduit extending between a region of the outlet of the second filtration module and a region of the at least one inlet of the first reaction module. Optionally, the apparatus comprises a plurality of inlet ports to allow the input of fluidic chemical component into the reaction channel or well. Optionally, the apparatus comprises at least one pump coupled to the at least one inlet of the first reactor module to drive fluid flow through the reaction channel or within the well. Optionally, the apparatus comprises at least one, valve, fluid flow gate, fluid flow port, heating element, fluid storage or retention reservoir provided in fluid communication with the fluid within the apparatus. Optionally, the pump comprises a syringe pump.

Optionally, the apparatus has a plate-like structure such that the first reactor module and the first filtration module are formed at least in part, as channels or recessed grooves provided on or internally at the plate-like structure. Optionally, the apparatus comprises a plurality of sensors positioned at different fluid flow regions of the apparatus.

Optionally, the sensors comprise any one or a combination of at least one temperature sensor, at least one flow rate sensor, at least one pressure sensor, at least one pH sensor, at least one flow volume sensor, at least one spectroscopic sensor, at least one optical sensor, at least one optical fibres or spectroscopic optic fibre.

Optionally, the apparatus comprises a control unit coupled to the at least one pump, valve, fluid flow gate, fluid flow port, heating element, fluid storage or retention reservoir and the sensors to control a characteristic of the fluid flow within the apparatus in response to a status of a physical, chemical or mechanical characteristic of the fluid determined by the sensors. Optionally, the control unit comprises a CPU, a processor, a PCB, a PLC, a handheld electronic device.

Optionally, the control unit comprises a control module comprising any one or a combination of the following: software; electronic components; a data storage utility; wired or wireless communication modules and/or ports; a visual display output; a user interface; at least one actuator to actuate any one or a combination of the at least one pump, valve, fluid flow gate, fluid flow port, heating element, fluid storage or retention reservoir and the sensors.

According to a further aspect of the present invention there is provided a fluid flow system for processing a fluid comprising: a plurality of the apparatus of claims herein, a final fluid flow outlet of each of the apparatus being coupled to a collection unit to combine the fluid output from each of the apparatus.

Optionally, the fluid flow system comprises control unit coupled to the at least one pump, valve, fluid flow gate, fluid flow port, heating element, fluid storage or retention reservoir and the sensors of each or a selection of the apparatus to control a characteristic of the fluid flow within the apparatus in response to a status of a physical, chemical or mechanical characteristic of the fluid determined by the sensors.

According to a further aspect of the present invention there is provided a method of processing a fluid using fluid flow apparatus comprising: introducing at least one fluid into a first reactor module via at least one inlet and allowing the fluid to flow through or within a reaction flow channel or well and outputting the fluid at an outlet of the first reactor module; driving at least a portion of the fluid from said outlet through a filtration module having a fluid filtration region via at least one inlet and outputting the fluid from the first filtration module at an outlet; wherein a reaction occurs in the first reactor module between at least two chemical components of the fluid and a filtration of the fluid occurs in the first filtration module.

Optionally, the method comprises driving fluid flow through the apparatus using at least one fluidic pump. Optionally, the method comprises recirculating at least a portion of the fluid from the outlet of the reactor module and/or the filtration module to a region of the at least one inlet of the reactor module via at least one recirculation conduit. Optionally, the method comprises monitoring a status of a chemical reaction occurring within the fluid between the chemical components using at least one sensor. Optionally, the method comprises controlling fluid flow through the apparatus in response to the status of the chemical reaction identified by the sensor. Optionally, the method comprises monitoring a physical, chemical and/or mechanical characteristic of the fluid within the apparatus using at least one sensor. Optionally, the method comprises controlling fluid flow within the apparatus in response to the identified physical, chemical and/or mechanical characteristic of the fluid by the sensor. Optionally, the step of controlling the fluid flow comprises controlling or deactivating at least one actuator, pump, valve, gate or port provided in fluid communication with the fluid within the apparatus. Optionally, the controlling the fluid flow comprises actuating or deactivating the at least one actuator, pump, valve, gate or port using a CPU, processor, PCB, PLC or handheld electronic device.

According to a further aspect of the present invention there is provided fluid flow filtration apparatus comprising: a first elongate fluid flow channel having an inlet; a second elongate fluid flow channel having an outlet; a permeable membrane positioned to partition the first channel from the second channel along the respective lengths of the first and second channels such that a permeate may pass from the fluid within the first channel via the membrane into the second channel along the lengths of the first and second channels.

Optionally, the first channel comprises at least one outlet; and wherein the inlet is provided at or towards a first lengthwise end of the first channel and the outlet is provided at or towards a second lengthwise end of the first channel. Optionally, the second channel may comprise at least one inlet; and wherein said inlet is provided at or towards a first lengthwise end of the second channel and the outlet provided at or towards a second lengthwise end of the second channel.

According to a further aspect of the present application there is provided a fluid flow system for the processing of a fluid comprising: a first reactor module having a reaction flow channel or well, at least one inlet and at least one outlet; a first filtration module having a fluidic filtration region, at least one inlet provided in fluid communication with the outlet of the first reactor module and at least one outlet; wherein the first filtration module comprises the fluid flow filtration apparatus as claimed herein.

According to a further aspect of the present invention there is provided a method of filtering a fluid using fluid flow filtration apparatus comprising: driving a fluid through a first elongate fluid flow channel from an inlet; forcing a permeate component of the fluid through a membrane extending along the first channel and into a second elongate fluid flow channel; and retaining a retentate component of the fluid within the first channel; wherein the membrane is positioned to partition the first channel from the second channel along their respective lengths such that the permeate may pass from the first channel via the membrane into the second channel along the respective lengths of the first and second channels.

According to a further aspect of the present invention there is provided a fluid flow system comprising: at least one reactor module having a reaction fluid flow channel or well, at least one fluid inlet and at least one fluid outlet; at least one flow driver to drive a flow of a fluid through the channel or well; a first sensor to measure any one or a combination of a pressure, a temperature or a pH of the fluid within the system; a second sensor to measure any one or a combination of a pressure, a temperature, a pH of the fluid within the system; a reaction status monitoring device to monitor a characteristic of a fluid within the system indicative of a status of reaction of at least two chemical components of the fluid within the system; a control unit to receive data from the at least one or a combination of the first sensor, the second sensor and the reaction status monitoring device and to control at least one characteristic of the fluid within the system.

According to a further aspect of the present invention there is provided a method of processing a fluid using fluid flow apparatus comprising: introducing at least one fluid into a reactor module via at least one inlet; driving the fluid to flow through or within a reaction flow channel or well using at least one flow driver and outputting the fluid at an outlet; measuring at least one or a combination of pressure, temperature or pH of the fluid within the fluid flow apparatus; monitoring a reaction status of chemical components within the fluid within the fluid flow apparatus; and controlling at least one characteristic of the fluid within the fluid flow apparatus using a control unit in response to at least one or a combination of the measuring of the pressure, pH, temperature and/or reaction status of the chemical components of the fluid.

### BRIEF DESCRIPTION OF DRAWINGS

A specific implementation of the present invention will now be described, by way of example only, and with reference to the accompanying drawings in which:

FIG. 1 is a schematic illustration of a continuous reaction and filtration apparatus suitable for RNA production;

FIG. 2 is a perspective view of a reactor and filtration module suitable for use in the apparatus of FIG. 1;

FIG. 3 is a plan view of a well or batch bioreactor module suitable for use in the apparatus of FIG. 1;

FIG. 4 is a plan view of a reaction channel-shaped bioreactor module suitable for use in the apparatus of FIG. 1;

FIG. 5A is a plan view of a filtration module suitable for use in the apparatus of FIG. 1;

FIG. 5B is an image of a membrane within the filtration module of FIG. 5A;

FIG. 6A is an illustration of a part of a filtration module having a region configured for centrifugal force separation of chemical components suitable for use in the apparatus of FIG. 1;

FIG. 6B is an image of a part of a filtration module having regions for the centrifugal force separation of chemical components;

FIG. 7 is a vertical tangential flow filtration (VTFF) module for use in the present fluid flow apparatus;

FIG. 8 is a magnified view of a region of the filtration module of FIG. 7;

FIG. 9 is a plan view of a spiral tangential flow filtration module;

FIG. 10A is a component of a filtration module according to specific implementation;

FIG. 10B is a schematic view of further parts of the filtration module of FIG. 10A;

FIG. 10C is a further illustration of parts of the filtration module of FIGS. 10A and 10B;

FIG. 11A is a plan view of a further filtration module formed as a two-layer microchannel TFF;

FIG. 11B is a schematic illustration of a filtration mechanism and filtration modules according to specific implementations;

FIG. 12 is a schematic view of a channel-flow bioreactor module;

FIG. 13 is a graph used in the calculation of the design of a fluid flow bioreactor and filtration system;

FIG. 14 is a schematic illustration of a reactor module coupled to a filtration module to form a fluid flow device;

FIG. 15 is perspective illustrations of a TFF module;

FIG. 16 is an image of microscopic analysis of the channel dimension of the TFF module;

FIG. 17 is a schematic illustration of a summary of a prototype building process;

FIG. 18A is a schematic illustration of a fluid flow system comprising a reactor module, a series of sensors, a reaction status monitoring arrangement and a control unit according to a specific implementation;

FIG. 18B is a schematic illustration of components of a reaction status monitoring arrangement suitable as a part of the fluid flow system of FIG. 18A;

FIG. 18C is a schematic illustration of a series of sensors, a reaction status monitoring arrangement and a control unit forming part of the fluid flow system of FIG. 18A according to a specific implementation;

FIG. 19 is a schematic view of an architecture of a part of a microfluidic flow system of FIG. 18 including a part of a spectrometer arrangement;

FIG. 20 is a schematic illustration of a serial communication program for the control system;

FIG. 21 is a graph of a relationship between an RNA yield and proportion of various reactants including NaCl, MgCl₂, NTPs etc;

FIG. 22 is a graph of the data analysis (RNA yield VS magnesium ion) and specific reaction time selection according to aspects of the subject invention;

FIG. 23A is a schematic illustration of a velocity profile analysis result simulation;

FIG. 23B is a graph of a velocity analysis graph;

FIG. 24 is a schematic illustration of the velocity in a tangential flow filtration channel within a filtration module;

FIG. 25A is a graph of the pressure drop in the bioreactor channel;

FIG. 25B is a graph of the pressure drops as multistep down;

FIG. 26A is a photograph of a mask for the manufacture of a fluid flow reactor and filtration apparatus;

FIG. 26B is a photograph of a continuous microfluidic flow reactor mask;

FIG. 27 is an image of continuous flow reactor sealed with Kapton tape;

FIG. 28 is an image of the complete set up using two Acrylic plates;

FIG. 29 is an image of the 3D printed mould after use;

FIG. 30a is a first part of a schematic illustration of an example embodiment of the present flow system integrated with a downstream formulation system using a modular flow reactor attached to a conventional fill and finish vaccine production line;

FIG. 30b is a second part of a schematic illustration of an example embodiment of the present flow system integrated with a downstream formulation system using a modular flow reactor attached to a conventional fill and finish vaccine production line;

FIG. 30c is a third part of a schematic illustration of an example embodiment of the present flow system integrated with a downstream formulation system using a modular flow reactor attached to a conventional fill and finish vaccine production line;

FIG. 30d is a graph of absorbance vs wavelength for an output solution of a flow reactor of the embodiment of FIGS. 30a to 30d evaluated using UV-Vis spectroscopy against a conventional batch protocol;

FIG. 31 is a photograph of an experimental fluid flow chemical compound synthesis platform;

FIG. 32 is a live plot spectrometer data (Intensities against wavelength) in Python;

FIG. 33 is an image of samples for use with a microfluidic flow reactor module and the continuous filtration system having different concentrations (A: 25 mM/L, B: 50 mM/L, C:75mM/L, D:100Mm/L);

FIG. 34 is a graph of a UV-Vis spectral analysis;

FIG. 35 is an image of a live plot pH data in Python;

FIG. 36 are graphs of Predicted Plot (On the left is the reaction with 1 mM dNTP and on the right is the reaction with 4 mM dNTP);

FIG. 37 illustrates graphs of a prediction Profiler for 1st and 2nd experiments (Upon is the reaction with 1 mM dNTP and below is the reaction with 4 mM dNTP);

FIG. 38 is a summary of various data of the fluid flow bioreactor and filtration (left is the reaction with 1 mM dNTP and right is the reaction with 4 mM dNTP LogWorth =-1og10(p-value));

FIG. 39 is a schematic illustration of a scaled-up fluid flow reactor and apparatus for the present system;

FIG. 40A is an illustration of a source code of absorbance data and real-time plot (Absorbance against Wavelength) in Python;

FIG. 40B is an illustration of a source code of live plot PH data in Python.

### DETAILED DESCRIPTION OF PREFERRED EMBODIMENT OF THE INVENTION

### Hydrodynamics Principles in Micro Porous Channels

Advances in microfabrication make it possible to build microchannels with micrometre dimensions. Since microchannels are usually integrated into these microsystems, it is important to determine the characteristics of the fluid flow in microchannels for better design of various microflow devices. The understanding of fluid behaviour in porous media, especially in microporous media that can be used for Lab on a chip design for synthetic RNA vaccine manufacturing is very limited due to the restriction of techniques. The porous medium is taken to be saturated with the fluid of interest in the sense that fluid-fluid interfaces do not form, and a single fluid prevails in the pore space. Let dp be the particle size and U, a velocity scale. (1) has been found to be applicable for Reynolds number of up to unity, namely

$$Re = \frac{ρ U {d}_{p}}{μ} < 1 \qquad \text{(1)}$$

The permeability of a porous medium is a property that depends on the pore size and the pore structure. Dimensional analysis shows permeability to be a function of porosity e and particle diameter dp; each representing the pore geometry and pore size, respectively. The Carman-Kozeny relationship connects these quantities empirically but with dimensional correctness as

$$K = \frac{{d}_{p}^{2} {ε}^{3}}{180 {\left(1 - ε\right)}^{2}} \qquad \text{(2)}$$

With the particle diameter expressed in meters, permeability has dimensions of m2. In applications, one can expect to have a length scale for the device itself, say L,and the ratio defined as

$$D a = \frac{K}{{L}^{2}} \qquad \text{(3)}$$

which is called the Darcy number. In many applications, the particle diameter is of the order of a fraction of a millimetre while the device length scale is around a meter or beyond. Eq. 2 can be properly stated as being applicable in the limit of Da<<1; Re<1. Bahrami et al. (M.Bahrami, 2006) developed a general model for prediction of pressure drop in microchannels of arbitrary cross section. Selection of the characteristic length is an arbitrary choice and will not affect the final solution. According to the model of Bahrami et al., pressure drop of laminar fully developed flow in arbitrary cross-section microchannels can be obtained from:

$$\begin{matrix}Δ P = 16 {π}^{2} μ Q {I}_{p}^{*} \frac{L}{{A}^{2}} \\ {I}_{p =}^{*} \frac{1 + {ε}^{2}}{12 ε}\end{matrix} \qquad \text{(4)}$$

where Ip* = Ip /A is the specific polar momentum of inertia of the microchannel cross section, Γ = 4(W+H) is the microchannel cross-section perimeter, L is the fully developed length, Q is the volumetric flow rate, ε = Aspect ratio (Width/Height) of the rectangular microchannels.

Reynolds number was calculated knowing the volumetric flow rate, Q, and the cross-sectional area, A, from

$${Re}_{\sqrt{A}} = \frac{ρ Q}{μ \sqrt{A}} \qquad \text{(5)}$$

Minor Losses, APmin: Other pressure losses associated with the measured pressure drop are inlet, exit, and bend losses. These losses are usually obtained from the traditional relationships used in macroscale. Phillips (ref) showed that the minor pressure losses can be obtained from:

$$Δ {P}_{m i n} = Δ {P}_{\text{in}} + Δ {P}_{o n} + 2 Δ {P}_{b} = \frac{ρ {Q}^{2}}{2 A} \left[{K}_{c} + {K}_{c} + 2 {K}_{b} {\left(\frac{A}{{A}_{t}}\right)}^{2}\right] \qquad \text{(6)}$$

Where A and Aₜ are the channel and connecting tube cross-sectional areas, respectively. K_{b} is the loss coefficient for the bend, and Kc and Kₑ represent the contraction and expansion loss coefficients due to area changes. Phillips recommended K_{b} to be approximately 1.2 for a 90 deg bend. Assuming equal cross-sectional areas for the channel and connecting tubes and maximum possible values for K_{c} and Kₑ, relative minor losses with respect to the measured pressure drop are negligible compared with the measured pressure drop. Hooman and Merrikh (M.Bahrami, 2010) have developed analytical solutions for flow and pressure drop inside porous channels as:

$$\frac{Δ P}{L} = \frac{μ Q s i n h \left(\frac{h}{\sqrt{K}}\right)}{K h \left[2 \frac{\sqrt{K}}{h} \left[- 1 + cosh \left(\frac{h}{\sqrt{K}}\right)\right] - s i n h \left(\frac{h}{\sqrt{K}}\right)\right]} \qquad \text{(7)}$$

and h, L and W are the depth, length, and width of the porous channel respectively. The cross-sectional aspect ratio, ε in the samples tested in the present study is 0.5. Therefore, instead of considering the whole rectangular cross-section, the sample can be modelled as a porous medium sandwiched between two parallel plates.

Choosing an appropriate cassette depends on the total sample volume, required process time, and desired final sample volume. Use the following equation to calculate the membrane area required for processing a sample in a specified time:

$$\text{A} = \frac{\text{V}}{\text{JXT}} \qquad \text{(8)}$$

Where,
A=Membrane Area (m2)
V= Volume of Filtrate generated (Litres)
J= Filtrate Flux Rate (Litres/m2/hour, LMH)
T= Process time (hours)

### Fluid Flow Simulation for the Study of a Prototype

The channel height and type of liquid effect on the performance of the microchannel. The computational fluid dynamic (CFD) FLUENT software was used for the simulation of microchannel. From the CFD, the velocity profile has been analysed for under and fully develop the region to study the fluid flow behaviour. The property of liquid influences the fluid flow inside the microchannel. We require the smallest kinematic viscosity and low surface tension that depend on select correct liquid.

The fluid flow plays an important role in leading small-scale devices to integrate with many applications because of their potential in chemical and biochemical engineering. The mechanism and fundamental of single-phase fluid flow have been review using experimental data for different types of liquid and surface roughness. Therefore, in a micro scale, the impact of liquid properties such as surface tension and viscosity is dominated. [Nawi, M. N. M., Manaf, A. A., Arshad, M. R., & Sidek, O. (2013). Numerical simulation of the microchannel for the microfluidic based flow sensor. Proceedings - 2012 IEEE International Conference on Control System, Computing and Engineering, ICCSCE 2012, 345-348.]

The Reynolds number is the ratio of inertial and viscous forces on fluid flowing in a channel (i.e., a ratio of the momentum of the fluid to the friction force imparted by the channel walls). A low Reynolds-number flow is a laminar or layered flow in which fluid streams flow in parallel to each other and mix only through convective and molecular diffusion. A high Reynolds-number flow is turbulent flow in which various-sized “parcels” of fluid exhibit motions that are simultaneously random in both space and time causing rapid mixing throughout the channel. The transition between the laminar and turbulent flow typically occurs at Re=2000 in internal flows [Schulte, T. H., Bardell, R. L., & Weigl, B. H. (2002). Microfluidic-technologies-in-clinical-diagnostics_2002_Clinica-Chimica-Acta. 321, 1-10]

Although a turbulent flow field is more complex than a laminar one, there are significant second-order influences. One and two-dimensional models depicting the time-dependent evolution of analysis distribution under such conditions have been improved. Confocal fluorescent microscopy experiments and three-dimensional numerical modelling can help to confirm the quantitative description of reaction-diffusion processes near the walls [Schulte, T. H., Bardell, R. L., & Weigl, B. H. (2002). Microfluidic-technologies-in-clinical-diagnostics_2002_Clinica-Chimica-Acta. 321, 1-10]

### Sensor Integration With the Flow System

In the study, a sensor integrated platform should be established to obtain and monitor data throughout the RNA transcription process. Gruber, P. [Gruber, P., Marques, M., Szita, N. and Mayr, T. (2017). Integration and application of optical chemical sensors in microbioreactors. Lab on a Chip, 17(16), pp.2693-2712.] proposed a flow diagram describing the path towards robust sensor integration in a fluid flow device, this diagram could be a guidance for the integration study. Various integrated platform systems have been designed and developed for real-time reaction monitoring [Wang, T., Kim, S. and An, J. (2017). A novel CMOS image sensor system for quantitative loop-mediated isothermal amplification assays to detect food-borne pathogens. Journal of Microbiological Methods, 133, pp. 1- 7.]. In Wang’s methodology, the complementary-metal-oxide semiconductor (CMOS) technology has shown great potential for integration and detection. CMOS image sensor works as an effective reaction and detection platform, enabling it to monitor real time photon changes as per the amplification process. The CMOS image sensor observed the photons and converted into digital units. In addition, UV-spectral studies, optical colour intensity detection and pH analysis were carried out to prove the efficiency of the CMOS [Wang, T., Devadhasan, J., Lee, D. and Kim, S. (2016). Real-time DNA Amplification and Detection System Based on a CMOS Image Sensor. Analytical Sciences, 32(6), pp. 653-658.]. Lopez-Huerta [Lopez-Huerta, F., Woo-Garcia, R., Lara-Castro, M., Estrada-Lopez, J. and Herrera-May, A. (2013). An Integrated ISFET pH Microsensor on a CMOS Standard Process. Journal of Sensor Technology, 03(03), pp.57-62.] has come up with an integrated ISFET pH microsensor on a CMOS standard process. The complete system was integrated in a 1.12 mm² silicon area; it presented a 59 mV/pH linearity, within a concentration range of 2 to 12 of pH level, making it a good alternative for biological or medical applications.

CMOS is a relevant choice, but considering the cost and time, it is difficult to achieve. More realistically, the integration scheme of each sensor has been reviewed. For pH sensor, a study of real-time feedback control of pH within microfluidics using integrated sensing and actuation [Welch, D. and Christen, J. (2014). Real-time feedback control of pH within microfluidics using integrated sensing and actuation. Lab on a Chip, 14(6), p.1191.] has been found. The system utilized an extended-gate ion-sensitive field-effect transistor (ISFET) along with an integrated pseudo-reference electrode to monitor pH values within a fluid flow reaction chamber. For temperature senor, a serpentine-shaped temperature sensor (linewidth: 50 mm) and heater (linewidth:400 mm) were integrated beneath the centre of the reaction chamber [Sun, H., Olsen, T., Zhu, J., Tao, J., Ponnaiya, B., Amundson, S., Brenner, D. and Lin, Q. (2015). A bead-based microfluidic approach to integrated single-cell gene expression analysis by quantitative RT-PCR. RSC Advances, 5(7), pp.4886-4893.]. Choi, K., Mudrik, J. and Wheeler, A. (2015). [A guiding light: spectroscopy on digital microfluidic devices using in-plane optical fibre waveguides. Analytical and Bioanalytical Chemistry, 407(24), pp. 7467-7475.] have presented a novel method for in-plane digital microfluidic spectroscopy. In this technique, a custom manifold aligned optical fibres with a digital microfluidic device, allowing optical measurements to be made in the plane of the device, which has provided an idea for optical detection. The preferred wavelength range of analysis for this invention is between 190 nm to 1000 nm. The peaks obtained are in the region of 260 nm.

### Methodology

### Continuous Flow Synthesis and Scaleup

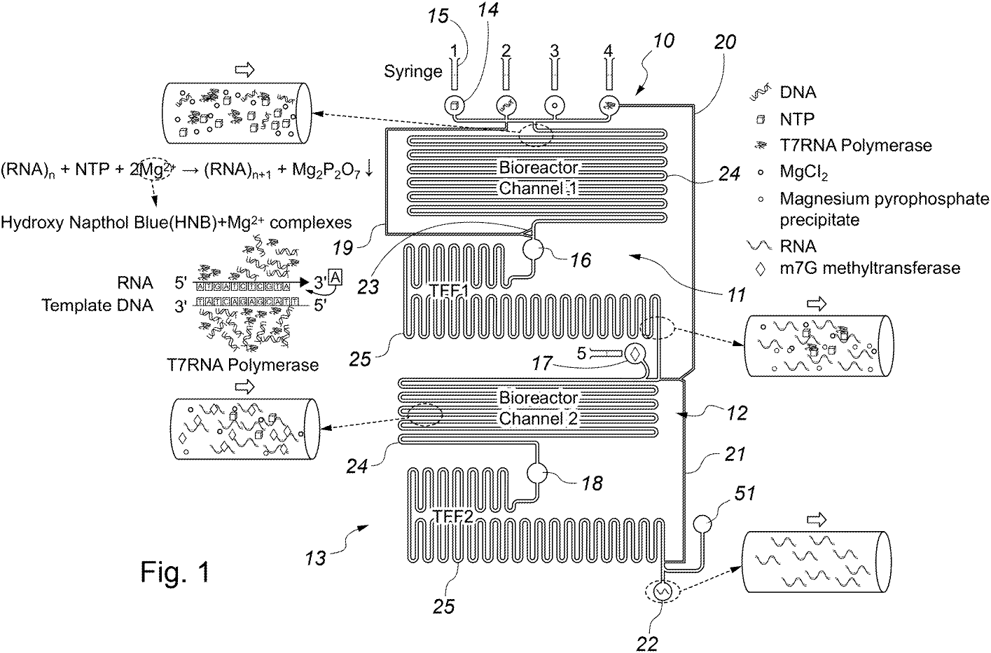

A schematic representation of the continuous RNA production process is shown in FIG. 1. As illustrated in FIG. 1, the fluid flow apparatus comprises a first bioreactor module 10, a first tangential flow filtration (TFF) module 11, a second bioreactor module 12 and a second tangential flow filtration (TFF) module 13. Each of the modules 10 to 13 are interconnected via respective channels or conduits. A plurality of fluid delivery input ports 14 are provided at the first reactor module 10 for the introduction of respective reactants into the first bioreactor 10. Each port 14 comprises a respective syringe pump 15. The first TFF comprises a respective input/inlet port 16, the second bioreactor module comprises a respective inlet/input port 17 and the second TFF module comprises a corresponding input/inlet port 18. Each of the port 16 to 18 may be provided with respective associated pumps or syringe ports for the introduction of fluid to drive fluid flow under pressure within elongate reaction channels 24 of each reactor and elongate filtration channels 25 within each respective module. A plurality of recirculating channels 19, 20, 21 interconnect various fluid flow regions (at or towards the respective inlets/outlet end regions) of the modules 10, 11, 12, 13 to provide fluid communication and return flow of reactants/reagent. The apparatus of FIG. 1 also comprises sensors, a reaction status monitoring device, a control unit and other associated electronic and actuator components (not shown) as described elsewhere herein so as to provide a fully automated and continuous fluid flow synthesis platform for the manufacture of chemical compounds including biological and non-biological species. The first bioreactor 10 at an outlet end of the elongate reaction channel 24 comprises a gate, 23, Gate 23 is configured to filter large molecular weight chemical components such as DNA from the fluid based on molecular size and/or molecular weight. The desired filtered fluid is then recirculated via channel 19 to an inlet region of reactor 10. This way, selected reagents and reactants can be recycled to provide a continuous or semi-continuous process.

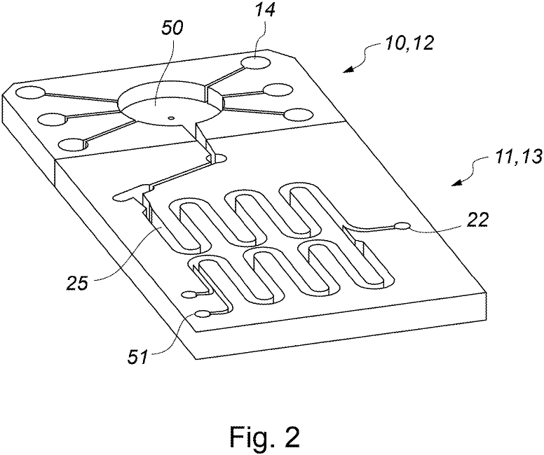

Referring to both FIG. 1 and FIG. 2, FIG. 2 illustrates a variation of the fluidic system of FIG. 1 in which reactor 10, in contrast to the elongate reaction channel 24 of FIG. 1, comprises a reaction well 50. As described with reference to FIG. 1, a plurality of injection ports 14 are coupled in fluid communication with well 50 for the delivery of the desired chemical compounds, reagents, solvents etc for the reaction. Filtration modules 11, 12 may comprises a series of extraction ports 51, 22 enabling the extraction of selected species, reagents, solvents from the filtration module and also collection of an end product for example at port 22 such as the fully capped RNA.

As will be appreciated, the specific details of the channel profiles, the use of ports, gates, inlets, outlets, pumps and fluid flow control actuators may be implemented with the arrangements as described herein.

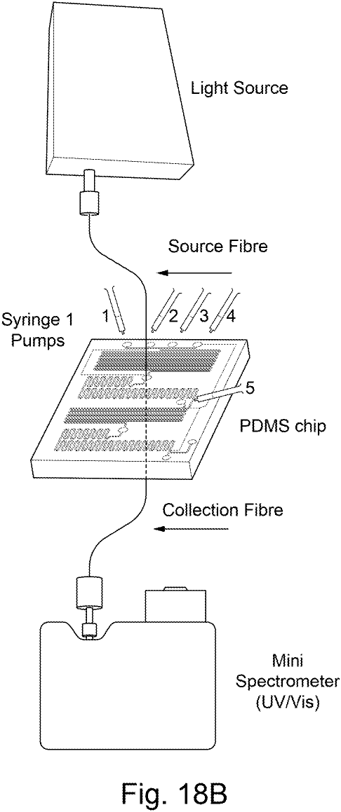

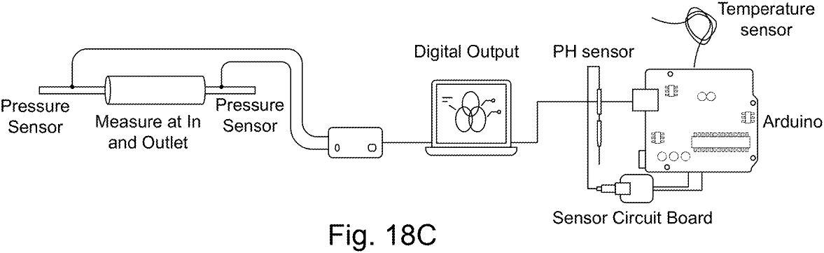

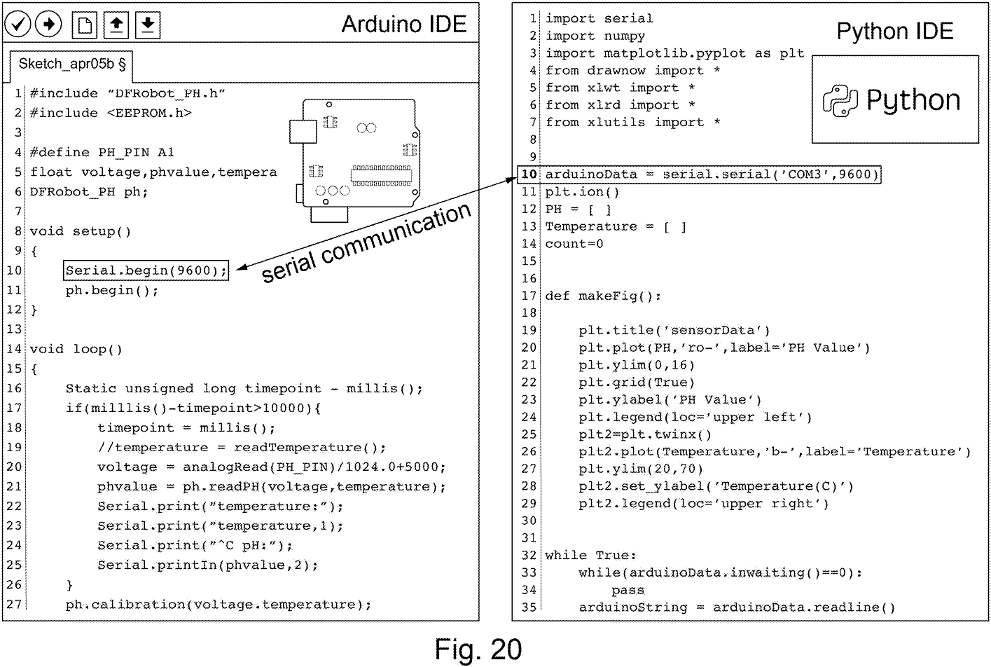

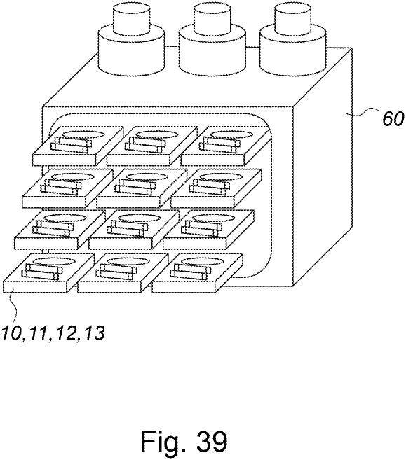

Referring to FIG. 39, a plurality of reactant and filtration systems as described referring to FIGS. 1 and 2 (and indicated generally as plate-like units 10, 11, 12, 13) may be connected together as a scaled-up fluid flow reactor and apparatus to form a fully integrated modular fluid flow reaction and filtration system for the manufacture of RNA-based therapeutics and prophylactic vaccines at scales sufficient for evaluation and clinical trials and ultimately production. In particular, suitable connection conduits provide fluid communication between the individual plate-like units 10, 11, 12, 13 such that the output of each unit (at output 22) may be combined to provide a total yield. The collective system of FIG. 39 may comprise a single control unit, multiple control units and respective sensors and reaction status monitoring utilities and units as described herein referring to FIGS. 18A to 20. With the parallel operation of a plurality of the fluidic devices of the present invention, the continuous manufacture of RNA-based therapeutics and prophylactic vaccines can take place. With this scaling strategy, one can reach manufacturing outputs at the scales required by the target markets, which can be the catchment area of a local hospital or the unit can be attached to a conventional fill-and-finish line at a manufacturer’s site. FIG. 20 is a schematic illustration of a serial communication program for the between an Arduino and a Python IDE control utility. In a possible embodiment an Arduino board could be used. In a preferred embodiment, a industrial edge compute SBC (Single Board Computer) node is used.

Using RNA synthesis as an example, during the process, a segment of DNA is copied into RNA by the enzyme RNA polymerase. The transcription mixture is composed of NTP (nucleoside triphosphate), DNA, MgCl₂ (reacts with P₂O₇⁻⁴ to generate precipitate, affecting productivity of RNA), T7 Polymerase (catalysis), which are injected into the first bioreactor channel by syringe pumps respectively. Hydroxy naphthol blue (HNB) is used as a colorimetric indicator for the titration of Mg²⁺ ions. Initially, the HNB and Mg²⁺ ions are combined in HNB—Mg²⁺ complexes. The decrease of Mg²⁺ ions results in colour change from violet to sky blue. The mixture stays in the 1st reactor 10 for approximately 6 hours, during which the RNA transcription reaction is going on all the time and RNA begins to be produced, accompanied with magnesium pyrophosphate precipitate (Mg₂P₂O₇). All the above components except DNA flow through the first tangential flow filtration (TFF) module 11, which has a large molecular weight (MW) cut-off membrane. The DNA is filtered back to the original inlet port 14(2) via recirculation channel 19 and kept in the 1st reactor, while others are filtered to next stage.

After the first filtration module, the above components are permeated downstream with the T7 Polymerase recirculated into inlet port 14(4). Then the m7G methyltransferase (5′ cap) is injected into a port 17 and flow into the second reactor 12 and channel 24, where the contents stay and react for 2 hours. The permeate enters the second TFF unit 13, which has a medium MW cut-off membrane. In this filtration stage, unreacted NTPs and 5′ caps are retained and recirculated (via 21 and 20) into the bioreactor 12 and/or 10. Finally, purified RNA is extruded at the outlet 22. Simultaneously, other components like Mg₂PPi, water, salts, are collected in another vessel or port 51 and optionally discarded or onward-processed. The output (capped RNA) may then be processed via further processing procedures (not described herein but familiar to those skilled in the art) to yield a desired vaccine.

An objective is to achieve continuous synthesis and purification to obtain RNA substance and achieve increased throughput of the system. It is necessary to add the raw materials into the reactor via parts 14 to compensate for the consumed molecules in order to maintain a continuous reaction of the process, which could be realised by regulating the actuator syringe pumps, and the feedback loop of material flow. Substantial improvements in RNA yield outputs might be possible by optimizing the reaction conditions (such as magnesium concentration, pH, temperature, reaction time).

### Set-Based Concurrent Engineering (SBCE) Methodology

Lean product development methodology has been chosen as the main framework to develop the design. Lean product development methodology is Set-Based Concurrent Engineering (SBCE). SBCE is a process where the product that wants to be developed is divided into different subsystems, allowing to develop a set of possible solutions for each of the subsystems in parallel. As the design evolves, the set of solutions for each subsystem gets narrowed down by knowledge-based decisions, using tools such as simulations, prototyping, testing or other gained knowledge. Following the SBCE methodology, the first essential step is to divide the product in different subsystems that can be developed individually. Considering the conceptual design of the systems that needs to be developed, there are four main identified processes that could be grouped in just two functionalities that will be developed: The bioreactor and the filtration process. Simultaneously, the system needs to be integrated with a control system, capable of controlling the pressure from the pumps as well as having sensors in strategic parts of the product in order to measure key parameters. Having mapped a conceptual design of the system, it has been possible to define different subsystems that can be developed in parallel. Table 1 shows the different subsystems, with a brief description and the corresponding level of innovation that has been defined for each subsystem.

TABLE 1

The modules of the flow system. These can be combined appropriately in any sequence required, according to the needs of the target therapeutic. For example, following the last filtration module there can be a further reactor/mixing step for the formulation of the RNA therapeutic substance to a therapeutic product.

| | |
|---|---|
| Subsystem | Description |
| Bioreactor 1 (10 - FIG. 1 ) | The considered volume for the bioreactor is 1 mL. There is one feeding channel for each of the raw materials, that goes into the reactor. The reaction needs to follow a continuous flow. |
| Filter 1 (11 - FIG. 1 ) | Several filters must be designed and developed in order to increase the RNA yield and to reuse some of the raw materials. This filter needs to separate and recirculate T7 Polymerase to the rest of the fluid. |
| Bioreactor 2 (12 - FIG. 1 ) | The reaction that goes on in the second reactor, concerns RNA and M7G Methyltransferase. It is essential that this reactor keeps the continuous flow of the process. |
| Filter 2 (13 - FIG. 1 ) | The second filter needs to achieve an RNA purity up to 99%. The M7G Methyltransferase needs to be cut off and taken and reused in the second reactor. |
| Control /Sensing system | A control system needs to be integrated with the whole system in order to support the different sensor/actuator devices. |

### Subsystem Design Specification Methodology Bioreactor

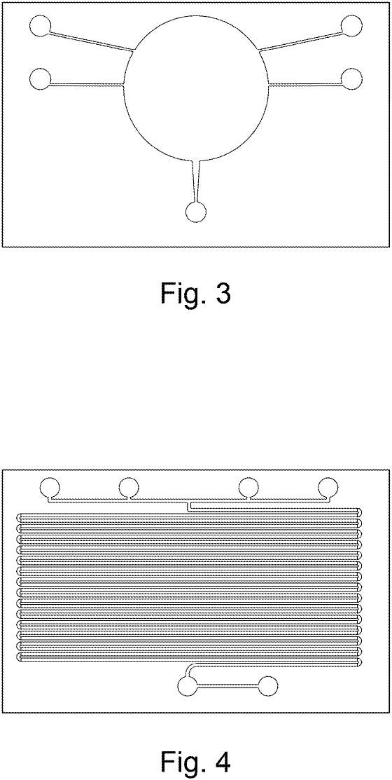

Regarding the bioreactor, two different designs have been developed. The first design is inspired by the conventional batch process used in chemistry for RNA synthesis. The design comprises four inputs 14 for each of the raw materials, that are connected to a main 1 mL chamber where the reaction takes place, by allowing sufficient residence time. Then the vessel can be selectively bled by the releasing of a microfluidic valve at the exit and fresh reagents introduced at the inlet. This would imply the device operates in continuous (perfusion) mode, in equivalence to a conventional batch system. Once the reaction is completed, the product is released into the output channel. The sensor evaluates whether it can proceed wot the next step of the process or rejected. FIGS. 2 and 3 show a design for a bioreactor.

This design needs a complex and well-coordinated control system to control valves integrated with the device. After the residence time is finished, the products need to be fed into the rest of the system.

The second design for the bioreactor is micro flow reactor shaped as a serpentine fluid conduit that allows mixing of the reactants and continuous synthesis of the target product as described in WO 2019/193346 A1 the details of which are incorporated herein by reference. This embodiment allows continuous synthesis by the control of fluid flow. In this design there are four inputs for the reagents, that are fed into the flow reactor continuously via a T-mixer. Reaction conditions and effectiveness is controlled by the net flow into the fluid conduit and the fluid velocity of the individual components through the T-mixer. These are controlled by setting the dispensing rate of the dispensing pumps with computer control. The product is formed at the time the mixture has reached the end of the serpentine-shaped flow bioreactor within a set residence time. At the output a feature of the device that acts as a small fluid holding well, temporarily slows down fluid flow to obtain an in-line measurement with the preferred method of analysis for the particular chemical solution such as in-line UV-Vis spectroscopy. The signal from the spectroscope is parsed and analysed in purposely built software by extracting certain features of the signal to determine if the desired product has indeed been formed by comparing against a reference vector. FIG. 4 is a schematic of the serpentine flow bioreactor.

### Filtration Modules

There are two types of filtration: Direct flow filtration (DFF) and tangential flow filtration (TFF). DFF consists in having the main flow perpendicular to the filter whereas TFF has a flow in a parallel direction to the filter. DFF has a much higher risk of clogging and it is demonstrated that while increasing the filtered volume, the filtrate flux rate is higher for the TFF that for the DFF. Moreover, typical applications for TFF are concentration, diafiltration and fractionation of biomolecules, as well as clarification and removal of cells; which are similar applications to the one present in this project. In other words, for this project’s application, TFF is the most effective option and consequently, the chosen type of filtration.

For the filtration module the TFF methodology was selected as a model. Several design solutions have been proposed in the research literature and commercial practice. Several other considerations have taken place for the present invention such as manufacturability and ability to integrate as a module with the whole system have been taken into account for this subsystem. The first filtration module design consists in a serpentine-shaped path with two channels at the same height, separated features purposely patterned on the device to act as a filtration membrane. X. Chen et al. (2007) [Microfluidic Chip for Blood Cell Separation and Collection Based on Crossflow Filtration. Sensors and Actuator B 130 (2008) (p.216-p.221). China.], have used in the past a TFF filter design for the separation of biological cells rapidly and reproducibly and at low cost compared to other solutions.

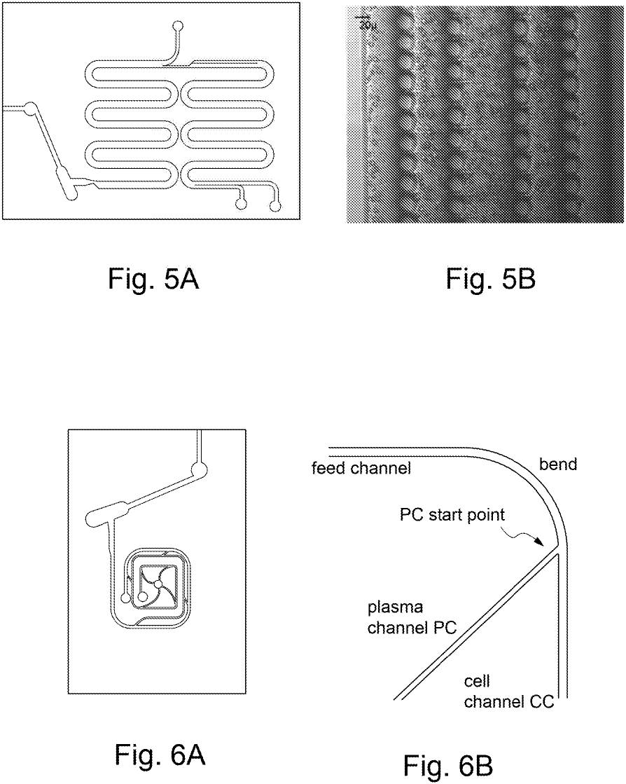

In the present invention uses a purposely selected membrane suitably sized for the separation of the biomolecules that are typically present in a solution following the synthesis of nucleic acids. FIGS. 5A and 5B show the filtration module, comprising the filtration features within the serpentine microchannels. The size and length of those channels has been purposely designed to allow the selection of the correct biomolecules in the filtrate and retentate flow streams. The manufacture of this module takes place using micro engineering techniques including photolithograpy and deep reactive etching of a glass or silicon substrate. These techniques are well understood, are industrially available, compatible with pharmaceutical regulation and are low cost for large quantity device production.

The second filtration module design does not need any filter membrane. It works with the principals of centrifugal force, which make some particles go faster than others if the fluid has a curved trajectory with a certain speed. So, having one bend microchannel that separates into two different channels, it is possible to have different sized particles go to different outlet channels [Blatter, C., Jurischka, R., Tahhan, I., Schoth, A., Kerth, P., Menz, W. (2005). Microfluidic Blood/Plasma Separation Unit Based on Microchannel Bend Structures. Proceedings of the 3rd Annual International IEEE EMBS. Hawaii.]. It is important to mention that this filtration method only works under appropriate conditions such as the fluid speed being over 1 [m/s]. With the right conditions, this separation technique can get efficiencies up to 90% and can simplify greatly the filtration process due to the lack of membrane. FIGS. 6A and 6B show the CAD design of this design solution.

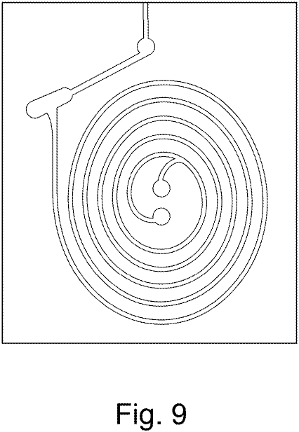

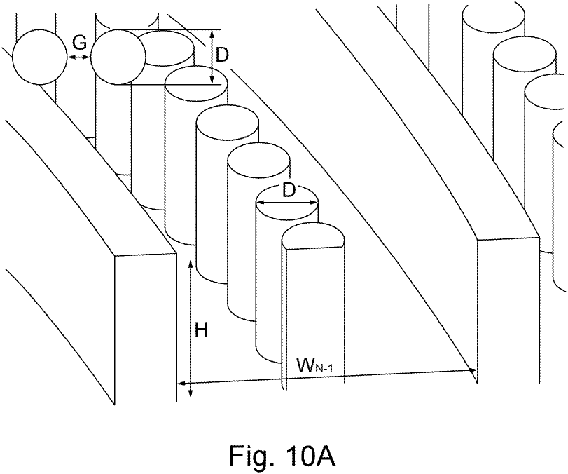

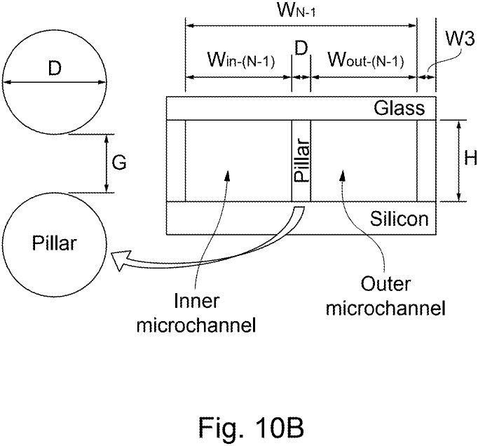

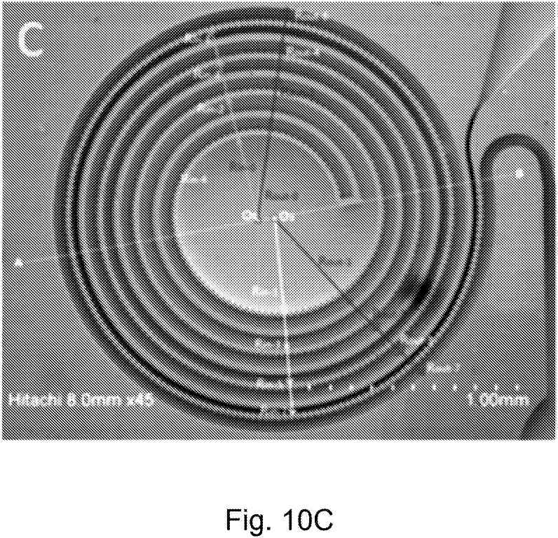

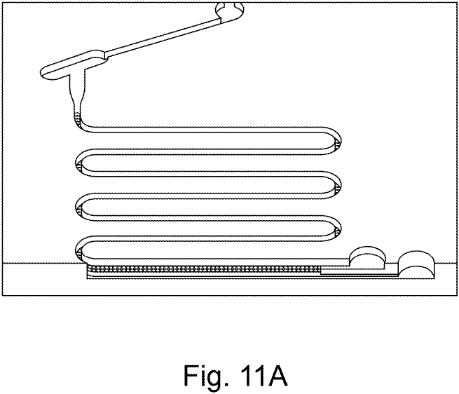

The third design is very similar to the first design module described above. The main difference is that the fluid pathway is spiral as described in WO 2019/193346 A1 the details of which are incorporated herein by reference. As Z. Geng et al. (2012) [Continuous Blood Separation Utilizing Spiral Filtration Microchannel with Gradually Varied Width and Micro-Pillar Array. Sensors and Actuators B 180 (2013) (p.122-p.129). China.] describe, this design adds the centrifugal effect to the conventional serpentine-shaped filtration module, described previously. This centrifugal effect increases the separation efficiency and reduces the risk of blockages. FIGS. 9 to 10C illustrate the design.

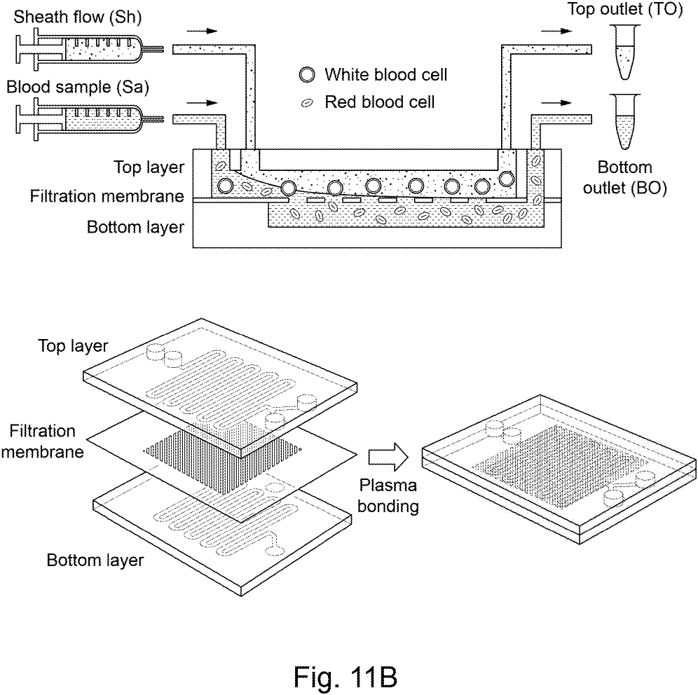

The fourth design for the filtration module uses a membrane that is sandwiched between two fluid streams. X. Li et al. (2014) [Continuous-Flow Microfluidic Blood Cell Sorting for Unprocessed Whole Blood Using Surface-Micromachined Microfiltration Membranes. Royal Society of Chemistry.] have used filtration membranes for blood cell separation. However, in the present invention, the filtration module uses two microchannel layers, which sandwich the filtration membrane to create the filtrate stream and the retentate stream which is recirculated back to the reactor module, while the filtrate stream continues is collected at the output and directed to a downstream module depending on the process requirements. The fluid comes from the reactor and goes into the TFF process through the top layer channel. Due to a pressure difference, some of the molecules in the fluid go through the membrane and into the bottom layer channel, whereas the molecules that are too big to go through the membrane stay on the top layer channel. This design solution offers good efficiency rates and most importantly, it offers a new perspective in terms of manufacturability. FIGS. 11A and 11B show a design of this solution.

The four embodiments of the filtration module described above rely on the control of pressure difference between flow streams and the method of separation used. However, in the fifth embodiment, the device combines digital micro and macro fluidics techniques and a commonly used protocols in molecular biology to separate and extract nucleic acids and DNA in particular. These protocols are two. One is charge based and the other attaches magnetic beads on the DNA or RNA for purification. For example in the present invention, magnetics beads such as the Invitrogen DNA binding beads (Thermofisher Scientific) attached to the DNA plasmids can be wither held in the reaction vessel using a digitally creted magnetic field as in the case of the bioreactor design of the present invention in FIG. 2 at the time the vessel is bled and refreshed with new solution, or forced through the filtration membranes in the filtration module designs described above and routed through the retentate channels back into the reaction module.

### Example Embodiment

Whilst all designs are modules in a plurality of configurations of a flow system for the manufacture of nucleic acid-based therapies, the serpentine-shaped filtration module represents a preferred example due to its ease and low-cost preparation. The entire process for the manufacture of prototypes for testing of the apparatus is explained further herein.

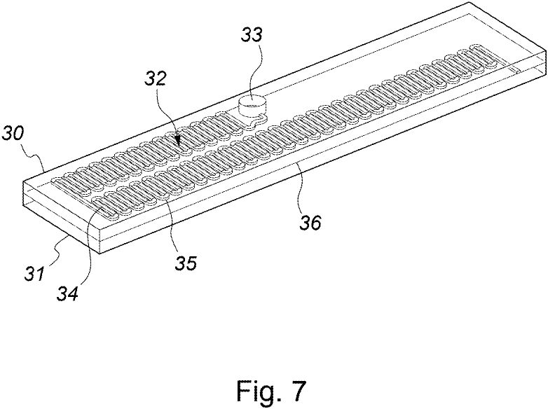

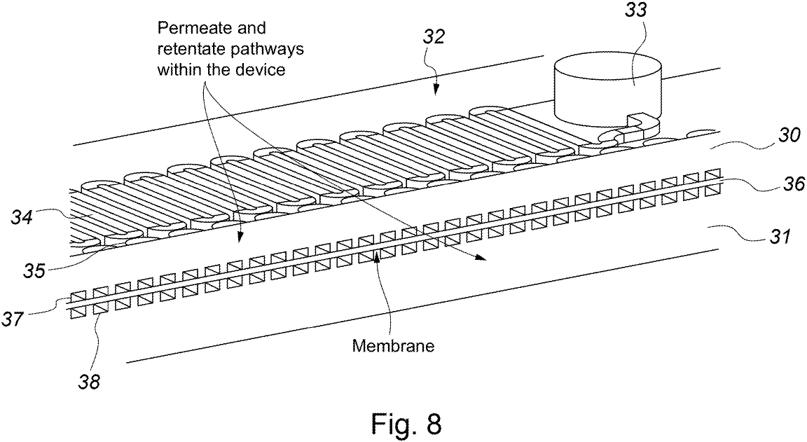

FIGS. 7 and 8, show the flow preferred filtration module which preferably comprises a first plate-like layer 30, a second plate-like layer 31 positioned with their main faces opposed to one another and sandwiching therebetween a membrane 36 to provide a lamella structure. Each of the plates 30, 31 comprises serpentine channels having straight sections 34 and bent or curved sections 35 to create respective fluid flow channels 37, 38 in direct contact with and at least, in part, defined by intermediate membrane 36. A collection, input or buffer reservoir 33 is provided at or towards respective lengthwise ends of any one of the first or second channels 37, 38. Accordingly, a fluid is capable of flowing through channel 37 of the upper plate with selected chemical components of the fluid having a smaller molecular size capable of defusing through the membrane 36 and into the adjacent fluid flow channel 38 of the second plate 31. Accordingly, TFF module of FIGS. 7 and 8 is configured for the separation of a permeate and a retentate differentiated by particle size (particle diameter) and/or molecular weight as will be appreciated.

### Fluid Flow Simulation Methodology for the Study of the Devices

As illustrated in Table 2, there are two different sizes of bioreactor channel that are required. Computational fluid dynamics (CFD) software has been used to analyse the assumption data in the microchannel. The microchannel requires a steady state flow rate.

TABLE 2

The calculation results of different sizes of bioreactor channel

| Inlet Flow Rate (µl/min) | Width (µm) | Height (µm) | Length (mm) | Viscosity (cP) | Density (g/cm³) | Pressure Drop (Pa) | Velocity (m/s) | Reynold’s Number | Flow Time (min) |
|---|---|---|---|---|---|---|---|---|---|
| 3 | 800 | 400 | 2500 | 3.5 | 1.06 | 149.9 | 0.00016 | 0.0252 | 260.42 |
| 3 | 1000 | 500 | 2500 | 3.5 | 1.06 | 81.9 | 0.00013 | 0.0269 | 320.51 |

FLUENT: Fluent is computational fluid dynamics (CFD) software which helps to address fluid flow problems. It uses the finite volume method to solve the governing equations for fluid and provides many different physical models such as laminar or turbulent, viscous or not viscous, compressible or incompressible. Geometry and grid generation is doing GAMBIT which is the pre-processor bundled with FLUENT.

A solution can be obtained by following these steps: Geometry; Mesh; Setup; Solution; Results. The channel is represented in a 2D CAD design. After, material properties and boundary conditions are set. Finally, the domain must be meshed. FLUENT converges the problem until the convergence limit is met or the number of iterations as specified is achieved.

### A) Geometry

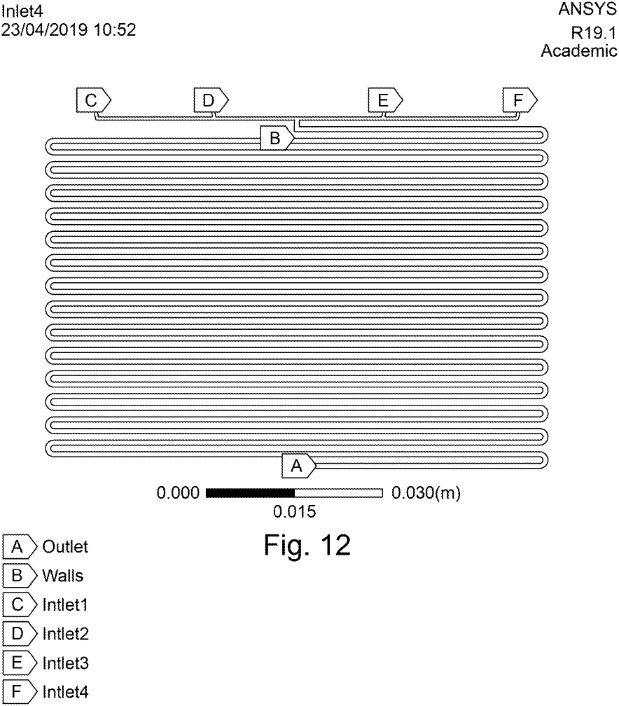

The geometry consists of walls, inlets and outlet boundaries, which is shown, in FIG. 12.

### B) Mesh

Coarse fine mesh types are available. Mesh density varies based upon the Refinement Factor, as illustrated in table 3.

TABLE 3

Coarse fine mesh types
Mesh Density Refinement Factor

| | |
|---|---|
| Fine | 1 |
| Coarse | 3 |

### Fluid Flow Modelling Using Computational Fluid Dynamics

Regarding the Reynolds number (Re = 0.0269) Laminar Flow has been selected. Air and water-liquid need to be selected as materials. The following material properties can be specified: Density and Viscosity.

The following boundary conditions have been assigned in FLUENT as shown in table 4.

TABLE 4

Boundary conditions assigned in FLUENT
Boundary conditions

| | |
|---|---|
| Inlet | Velocity inlet |
| Outlet | Outflow |
| Wall | Wall |

### C) Solution

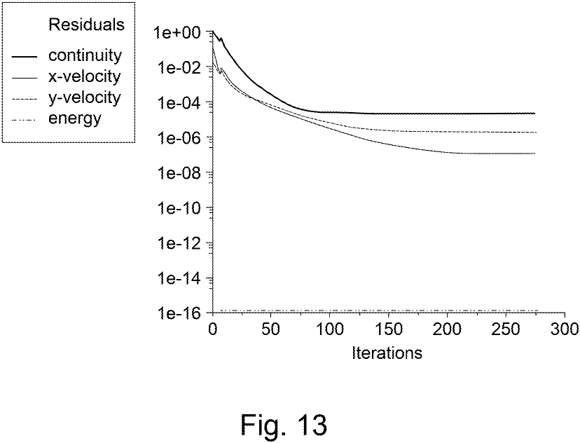

The mesh is exported to FLUENT along with the physical properties and the initial conditions specified. When the solution is converged or the specified number of iterations is met, FLUENT exports the data. As table 5 shows, the total flow time is 14400 seconds, which is 4 hours so that every 60 seconds CFD software record data which needs to repeat 240 times, Then the CFD software analyses the velocity of X and Y directions, energy and continuity. Finally, after 38 iteration the results have converged as shown in FIG. 13.

TABLE 5

Input and Output Data FLUENT
Input and Output Data

| | |
|---|---|
| Time Step Size(s) | 60 |
| Number of Time Steps | 240 |
| Flow time(s) | 14400 |
| Converged | 38 |
| Total iteration | 276 |

### Prototype Construction

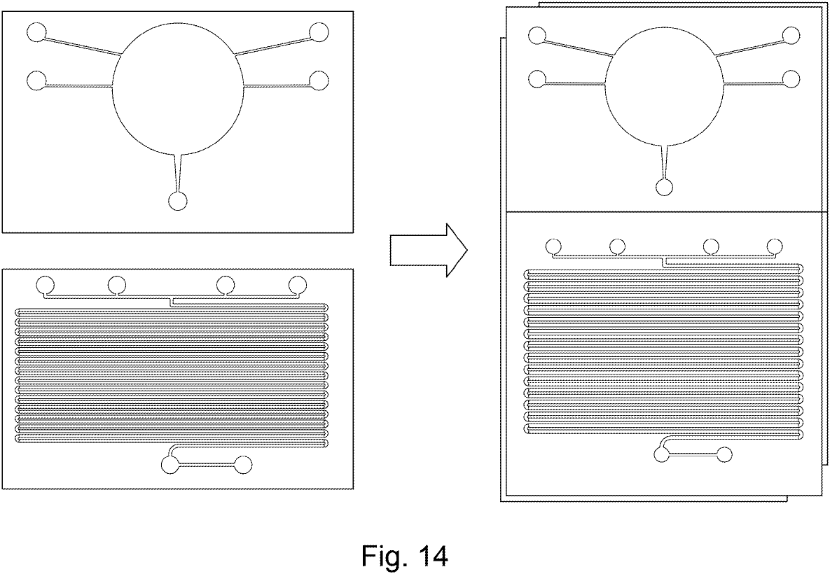

To build the prototype different steps have been followed. Moulds have been created first and the chips have been casted from them. The different sub systems have been built separately in order to test each design. Experiments will be done on each sub system to validate the design. Different versions of the moulds have been edited. The design evolution is represented in FIG. 14.

After experiments made on each mould some improvements have been made between the two versions.
The two templates have been regrouped into one making the all preparation chip building process easier.
The cross-section area of the channels has been increased from 0.4 mm×0.8 mm to 0.5 mmx1 mm avoiding the walls of the channels to collapse when testing the chip
Extra space between the channels (0.5 mm to 1 mm) and between the mould’s border and the features have been added to improve the quality of the chip and avoid any accidents when removing the PDMS mask from the mould.
Finally, some walls have been added to the borders of the moulds to improve quality of the whole PDMS chip when casting.

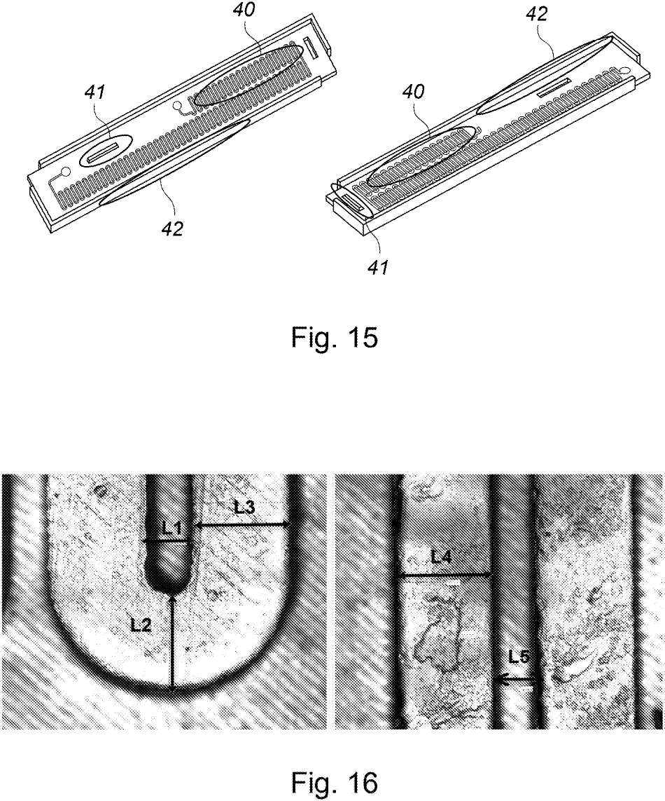

Regarding the Vertical TFF device the top and the bottom layer have been edited separately to create two different layers which will be sealed together with the filtration membrane in the middle. FIG. 15 illustrates the design of two layers. Table 6 explains the different features of the two designs.

TABLE 6

Feature explanation of the two designs

| | |
|---|---|
| Features | Explanation |
| Main vessel 40 | The main vessel has a cross section area of 0.5 mmx1 mm. The two moulds have identical channel. The two channels are reverse to aloud to have same channel when sealing the two layers one on top of each other. |
| Male and female plugs 41 | On both masks a female and a male plug have been added to help the positioning between the two layers during the assembly. |
| Walls 42 | For the same reason as the reactor walls have been added around the moulds to improve the quality of the chip. |

### Soft Lithography & SLA 3D Printed Moulds

Microfluidics chip requires precision, accurate method and technique in order to make reactions or filtration work as expected. The most common method that is used for microfluidics chip is Soft Lithography. In this case Soft Lithography consists in creating a mould where PDMS will be casted from [Kamei, K. ichiro, Mashimo, Y., Koyama, Y., Fockenberg, C., Nakashima, M., Nakajima, M., ... Chen, Y. (2015). 3D printing of soft lithography mold for rapid production of polydimethylsiloxane-based microfluidic devices for cell stimulation with concentration gradients. Biomedical Microdevices,]. The mould is 3D printed from the CAD design of the wanted part. Although different techniques can be used for 3D printing, in the project case Stereo Lithography has been selected.

With the different moulds designed, the parts were sent to be 3D printed using SLA. SLA printing allows to build complex shapes with a precision of 10 µm in approximately 24 hours. Regarding the utilisation and the different shapes and features that the moulds required, the best resolution was set on the SLA machines to optimize the resolution and precision of the printing. The first version of the mask was analysed through a microscope to compare the size between the CAD file and the 3D printed part. FIG. 16 illustrates two microscopic analysis of the channel dimension.

TABLE 7

Microscopic analysis value of channel dimension

| | | |
|---|---|---|
| Measurement | Value | CAD Dimension |
| L1: Distance between channels | 437 µm | 400 µm |
| L2: Channel width at the 90° bend | 902 µm | 800 µm |
| L3: Channel width in regular section | 902 µm | 800 µm |
| L4: Channel width in regular section | 910 µm | 800 µm |
| L5: Distance between channels | 440 µm | 400 µm |

Finally, the moulds have been printed in two different materials. The two are photopolymer resin with one being a high temperature resin. The main difference of these two materials are the maximum temperature before heat deflection. The first one can reach 50° C. when the second one can go up to 250° C.

### Filtration Membrane Selection

The selection of specific filtration membrane has been managed in two steps. The first steps consisted in finding the molecular cut-off size of the two different membranes for the two filtration stages. In fact, each filtration stages are dealing with different molecules with different molecular weight. As shown herein with the conceptual process some of the reagent’s molecules need to be filtered and recycled back to their respective bioreactors due to their high cost. Table 8 details the different molecules and their size or molecular weight.

TABLE 8

Molecular weight information

| | | |
|---|---|---|
| Molecules | Molecular weight and spherical dimension | Filtration stages |
| DNA | 10 MDa → 30 nm | End of Bioreactor 1 |
| RNA | 5 MDa → 23 nm | Final purified product |
| T7 polymerase | 99 kDa → 6.3 nm | Vertical TFF 1 |
| M7g + Methyltransferase | 300 kDa → 9 nm | Vertical TFF 2 |

Regarding the two vertical TFF stages, the first one is dealing with T7 polymerase whereas the second vertical TFF is dealing with M7g+Methyltransferase. Considering the different molecules sizes, the selected filtration membrane needs to have a molecular cut-off size three times smaller than the molecules that needs to be kept, in this scenario it is the RNA molecules [General Electric, 2014). In addition, the filter needs to be large enough to let the recycling molecules go through it. Given all this information the molecular cut-off size of each membranes has been allocated as followed:
Vertical TFF 1 ➔ Molecular weight cut-off membranes of 300 kDa
Vertical TFF 1 ➔ Molecular weight cut-off membranes of 500 kDa

The second step of the process was to select an appropriate membrane supplier. Therefore, because of time restriction, 1 mx1 m flat sheet membrane from the company Synder filtration have been selected. Three types of membranes have been ordered with different pore size: 300 kDa, 400 kDa and 500 kDa allowing to make different test and experiments and analyse the filtration effectiveness of each membrane.

### PDMS Mask and Casting

With the mould printed and ready, Polydimethylsiloxane (PDMS) microfluidics can be casted from it. The product used for creating PDMS chip is Sylgard 184 silicone elastomer. This product is delivered with two elements, one silicone elastomer part and a curing agent responsible of the elastomers’ molecules crosslinking, which will solidify the elastomer while keeping casting properties. However, some preparations are required before applying PDMS over the mould. Table 9 shows the curing temperature with their curing time.
Silicone elastomer is mixed with curing agent with a mass ratio of 10:1 (Silicone/curing agent) for 10 minutes in a beaker.
After the mixing, the air bubbles in the mixing need to be removed with the help of a desiccator. Repeat this operation until all the bubbles have disappeared.
If small bubbles are remaining let the beaker rest. The bubbles will pop by themselves.
When the mix is ready, applied it on the 3D printed mould and let it rest for 10 to 20 minutes

Put the mask with the PDMS on top in the oven or let it rest at ambient temperature. • Table

TABLE 9

Curing temperature and time

| | |
|---|---|
| Curing temperature | Curing time |
| 22° C. | 48 H |
| 60° C. | 5 H |
| 100° C. | 35 min |
| 125° C. | 20 min |
| 150° C. | 10 min |

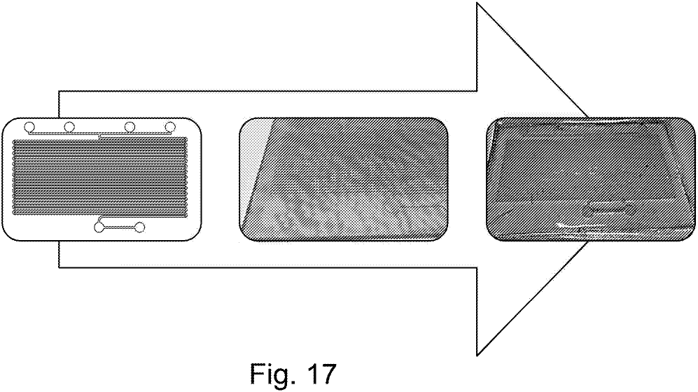

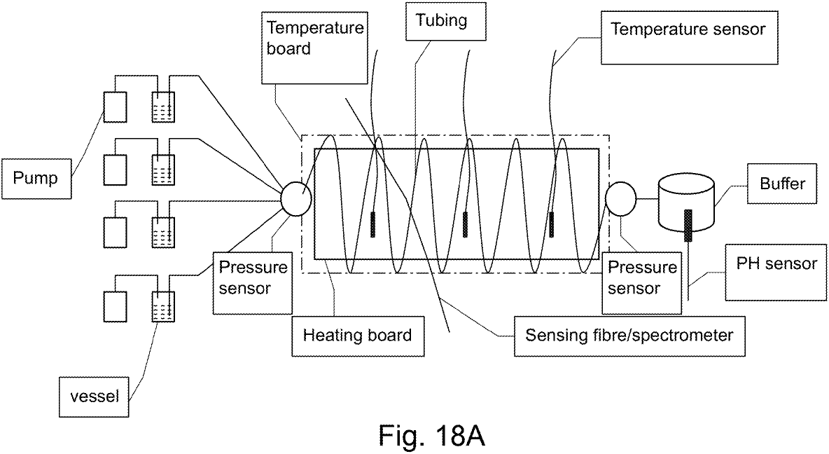

Different temperatures have been tested to compare PDMS curing properties and aspect. However, the first mould used in this process were not able to support high temperature. It is why the ambient temperature curing have been tested over a week end as well. Finally, with the different features casted on the PDMS mask, this one needs to be sealed on the open side. Two different methods have been used to seal the mask to allow different result from tests (FIG. 17 is a summary of the different steps of a prototype building process to create a continuous flow bioreactor system):
The first one consisted in sealing the PDMS masks with another flat layer of PDMS. This method allowed to add extra thickness of the whole mask and aided the integration of tubing and fittings to it.
The second method consisted in sealing the PDMS masks with Kapton tape on the open side. This method allows a perfect sealing between the features of the PDMS mask and the tape. However, the integration of tubing and fitting will be more complicated as the thickness of the mask is reduced.

### Sensing Methodology

The main sensing system is designed combining the feature of the continuous flow microfluidics system and the reaction. Firstly, the sensing system of the microfluidics chip is limited by the chip size and the characteristics of the factors that need to be detected. Furthermore, to select suitable sensors for obtaining accurate and real-time detection results of the chip is another key point. The sensing system of the micro-factory comprises spectrometer, pH sensor, and pressure controller and sensors. The theories behind the spectrometer are discussed by L. Zhu, (2005) [, Nuno Miguel Matos Pires, (2014) and Kihwan Choi, (2015). The theory behind the pressure controller and sensors have also been explored by Yung-Shin Sun, (2016).

### Material Selection

### A) Spectrometer

The spectrometer (USB2000+ Miniature Fiber Optic Spectrometer, Ocean Optics inc, UK) can store 1000 full spectrum per second, and its detection range is from 190 to 1100 nm wavelength. In addition, to use the official software, programming with open source packages in Python according to the characteristics of the sensing system is the main. This spectrometer is suitable for monitoring the continuous reactions.

### B) pH Meter

The measuring range of pH meter (HI-1093B pH Electrode, Hanna Instruments Ltd, Bedfordshire UK) is 0 to 13. In order to integrate the pH meter with the microfluidics chip, the size of pH meter is limited due to the design specifications. The diameter of measuring cell on the microfluidics chip is 5 mm2, and the body of the pH meter is 3 mm diameter.

### C) Pressure Controller and Sensors

The CFD analysis of the microfluidics chips suggests that the pressure of the bioreactors and TFF sections is 0.27 Bar. The pressure sensor (MPS Microfluidic High Precision Pressure Sensor, ELVEFLOW, Paris France) can provide 5 ranges of measurement, from 70 mBar to 7 Bar. It also allows to detect an ultra-small internal volume to 7.5 µL, which can apply to the 0.5 mL volume of the microfluidics chip. The flow rate regulation of this sensor is sensitive and responsive, so it is appropriate for the real time monitoring of the minor changes in flow rate. The mating sensor reader (Sensor Reader, ELVEFLOW, Paris France) can provide a high-speed capability and is easy to integrate into the chips, which makes the measurement simple and feasible.

### D) Pressure Supplier

Syringe pumps (C3657 C-Series Syringe Pumps, Tricontinent, CA USA) are used as pressure supplier to feed the reactant materials into the microfluidic chip. The stroke speed of the pumps are 1.2 seconds to 100 minutes per stroke, and pump resolutions are 3,000 steps of standard mode and 24,000 steps of high-resolution mode. The reaction time of the system is 4 to 6 hours. Also, these syringe pumps can provide stable flow rate in the microfluidic systems. The pressure of 5 inlets for injecting the responsive components can control the proportions of reactants to achieve an economical and efficient yield of RNA vaccine.

### Method

The key parameters of the reactions need to be monitored are RNA and Mg2+ concentration, pH value of the reactions and the temperature. At the same time, inlet pressure needs to be controlled, in order to achieve the appropriate proportion of RNA yield, and to adjust the flow rate of the mixture solution in the continuous flow channel for a proper reaction time. There are 3 detection points on the microfluidics chip, two points are used for quantitation of RNA and Mg2+ and the other for pH value measurement. Also, the pressure sensors are fabricated outside the chip. The concentration level change of Mg2+ and RNA can be used to detect the rate of reactions. The method of converting absorbance to concentration is a common way to accomplish the quantitation of RNA and Mg2+. UV absorbance measurements at 260 nm is a gold standard of RNA quantitation, and the Mg2+ quantitation can be obtained with absorbance measurements at 680 nm. The absorbance measurements of RNA and Mg2+ can be converted to concentration using the Beer-Lambert law. In the design, PDMS is used to make the bioreactors, which is sufficiently transparent. This makes the effective RNA detection viable.

In the reaction, the corresponding thermostable DNA polymerase is the main source of pH value. The pH of 8.3 to 9.0 can give optimal results in the system. Also, increasing the pH value in the reaction can help to stabilize the DNA template and enhance the transcription results. The recombinant enzyme activity is significantly influenced by the pH of the assay. For integration of devices, Arduino micro controllers (ARDUINO UNO REV3, Arduino, UK) have been used at the core for data collection.

### Integrated Experimental Platform

A schematic illustration of the present fluid flow reaction system is illustrated in FIGS. 18A to 18C. The system comprises a plurality of sensors adapted for the measuring of various characteristics of the fluid flowing within the fluid flow system including pressure, temperature, pH, flow rate, flow volume etc. Also incorporated within the system is a plurality of valves, injection ports, pumps in particular syringe pumps, gates etc. to control the flow pathway of components of the fluid within the apparatus. For example, components of the fluid may be recirculated from an outlet port at the lengthwise end of a bioreactor and/or filtration module to return to an inlet port of an upstream bioreactor and/or filtration module as desired. The system further comprises a control unit that typically comprises a CPU, microprocessor or other suitable electronic processor device. According to a specific embodiment, the processor is integrated into a PCB or PLC. The control unit is provided with a user interface, sensing data storage utility, software, visual display device, communication ports and/or modules, at least one analogue to digital converter and/or a data source library. Such components may be utilised by the control unit in combination with the sensors, injection ports, pumps, valves etc. to control continuously and automatically fluid flow within the fluid flow system. Accordingly, a continuous reactor system is provided for the continuous input of reagents to enable the continuous output of desired reactant products. The present system is advantageous to recirculate and reuse reagents, solvents etc. to minimise waste and to maximise efficiency. Once the facilities are ready, the layout of them will be designed and the experiment environment will be set up. Necessarily, the PDMS chip ought to be fixed and clamped by a fixture. Then the locations of other instruments are determined accordingly.

At the beginning, 4 syringe pumps were used to put pressure into the 4 input vessels respectively. Tubing and fittings are generally used to transport small volumes of samples to the PDMS chip. Another pump will be connected to a second vessel, which is located at the start point of the second bioreactor (end of 1 st TFF), making it possible to inject the m7G methyltransferase and regulate pressure in the chip, the use of a pressure sensor with a feedback loop will increase the responsiveness of the flow control significantly. The pressure sensors are considered to measure at inlet and outlet of the channels. The target is to keep the syringe pumps working while still being able to ensure a constant pressure in the device.

To implement the concentration detection, UV/Vis light should go through the detection chamber, where is at the end of the first bioreactor (serpentine channel), are transmitted through optical fibres. Two miniature optical fibres which match the specific wavelength needs are installed vertically to each other: a source fibre that is connected to the light source, and a collection fibre connected to the mini spectrometer. During the continuous bio-reaction, a pH sensor is planned to put into a buffer to measure the PH value, while temperature sensors can be placed on the board with a heater on the other side to adjust the temperature. The pH sensors and temperature sensors are equipped with BNC connector, which can be connected with Arduino. Thus, the data could be obtained from those sensors.

### Data Acquisition and Monitoring

### Spectrometer Data

The Lambert-Beer law describes the linear relationship between the analyte concentration and light absorbance of specific wavelength:

$$\text{Absorbance} = {\text{log}}_{10} \left(\frac{{I}_{0}}{{I}_{1}}\right) = ∈ ⋅ L ⋅ C \qquad \text{(9)}$$

Where: I₀ is the initial intensity of the light source, I₁ is the light intensity after it passes through the sample, ∈ represents the wavelength-dependent molar absorptivity coefficient with units of M⁻¹ cm⁻¹, L is the path length, and C is the analyte concentration.

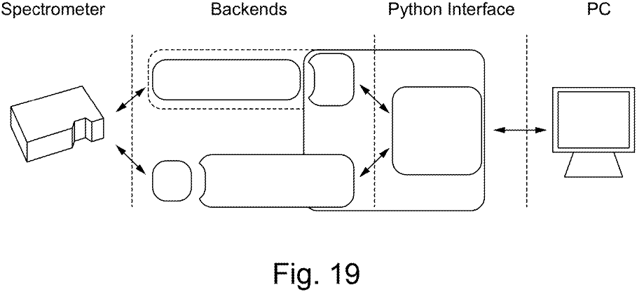

Therefore, the expected spectrometer data is exactly the absorbance spectrum. The Ocean Optics spectrometer has provided an easy way to access the data from Python. That is the Python-Seabreeze package, which wraps the Seabreeze library to communicate with the spectrometer. It provides a fully working and tested reference implementation of the Ocean Optics USB interface, which means the spectrometer data can be read and monitored using python. The connection between python and spectrometer is shown in FIG. 19.

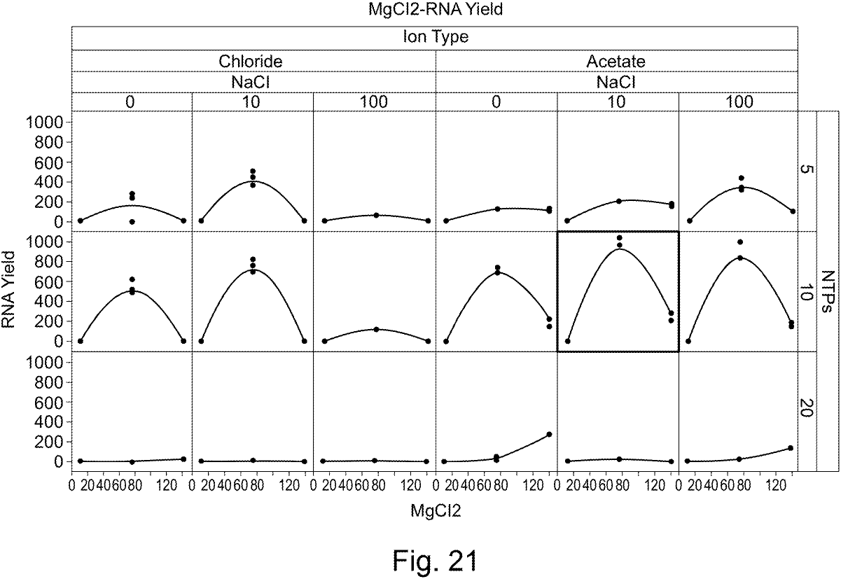

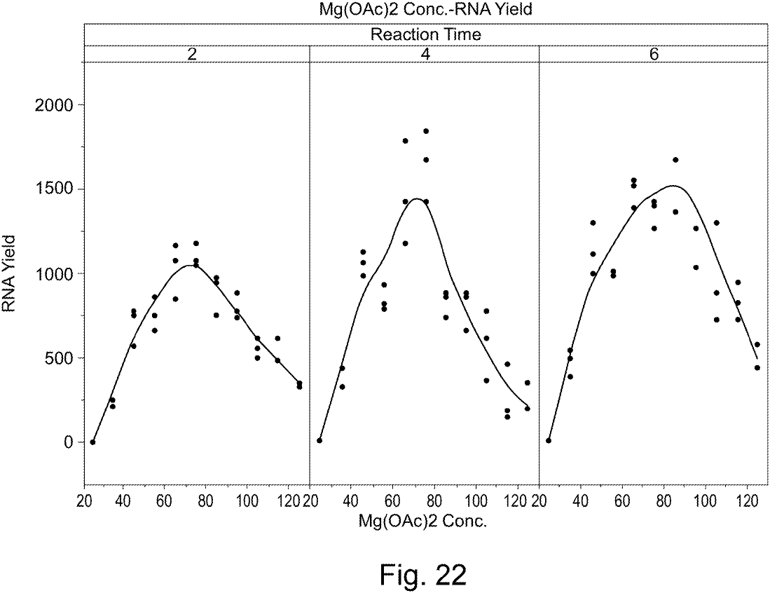

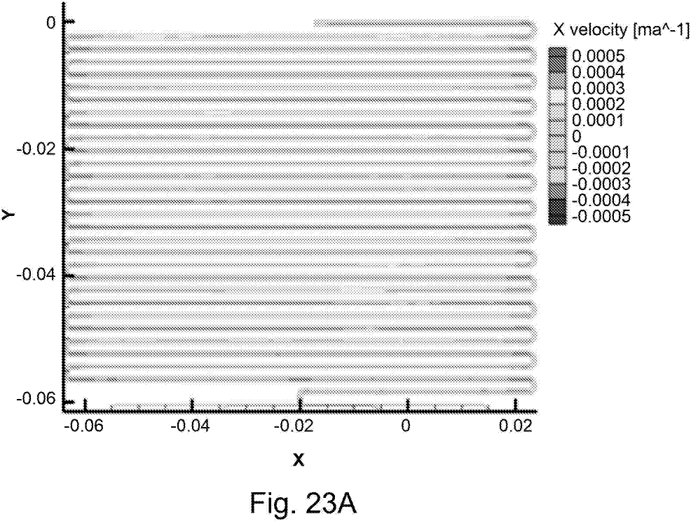

Once the pH sensor has been connected with Arduino through BNC connector, the sensor data can be obtained, and the real time data can be checked on serial monitor. The code for this is shown in FIG. 19. The same mechanism is used to obtain the temperature sensor data. Five full factorial experiments were conducted focussing on the influence of the different proportion of reagents as well as reaction time and the proportion of Magnesium icon. The experiments were operated based on the fed-batch system with a volume of 500 µL. Background experiment data is shown in below. Several conclusions can be drawn from the data. FIG. 21 shows the maximum output coming from the mixture of 10 mM NTPs, 10 mM NaCl, 75 mM MgCl₂ with Acetate ion, which is around 900 mM RNA yield, which could be regarded as the optimum combination. The optimum reaction time for RNA yield is shown in FIG. 22. The RNA yield can reach a highest level when the proportion of Mg(OAc)2 is approximately 80 mmol. After four hours reaction, the RNA yield achieves stability at around 1800 mM. The optimum combination of the reaction condition depends on the four main factors: the proportion of MgCl₂, NaCl and NTPs as well as the reaction time. For the further experiment, the proportion of MgCl₂, NaCl and NTPs need to be monitored to keep it at a certain level for getting the maximum yield of RNA. The most remarkable difference of the experiments between Imperial College and Cranfield University is the reaction system design. Due to the manufacturing consideration, continues follow system is designed for the bioreactor process instead of the fed-batch reaction system. To test the continuous flow system, the experiment is designed based on the previous experiments conducted by Shattock Group at Imperial College.

Three levels are chosen for four factors (table 10) to test the optimum proportion for RNA yield in a continuous flow system. The selected factors refer to the result of fed-batch system experiments. Additionally, the values of DNA and T7 Polymerase are the same for the recycle using reason. With these factors, the new design of experiment is listed as table 11. The experiment aims to find the relationship between RNA yield and four factors and to identify the trend of reaction.

TABLE 10

Factors and Levels selected for flow synthesis experimentation.

| No | Factors | 3 Levels | | | Response |
|---|---|---|---|---|---|
| 1 | MgCl2 | 70 | 75 | 80 | RNA Yield |
| 2 | NTP | 2 | 4 | 6 | |
| 3 | DNA | 8 | 10 | 12 | |
| 4 | T7 Polymerase | 8 | 10 | 12 | |

TABLE 11

Design of Experiments table for flow synthesis experimentation.

| Experiment Run | Factors | | | | Response |
|---|---|---|---|---|---|
| | MgCl₂ (mM) | NTPs (mM) | pDNA (mM) | T7 | RNA Yield |
| 1 | 1 | 0.25 | 0.05 | 0.05 | |
| 2 | 1 | 0.5 | 0.1 | 0.01 | |
| 3 | 1 | 0.1 | 0.05 | 0.01 | |
| 4 | 1 | 0.5 | 0.1 | 0.1 | |
| 5 | 1 | 0.25 | 0.05 | 0.1 | |
| 6 | 2 | 0.5 | 0.01 | 0.1 | |
| 7 | 2 | 0.5 | 0.05 | 0.05 | |
| 8 | 2 | 0.1 | 0.1 | 0.01 | |
| 9 | 2 | 0.1 | 0.1 | 0.1 | |
| 10 | 2 | 0.25 | 0.1 | 0.05 | |
| 11 | 2 | 0.1 | 0.01 | 0.01 | |
| 12 | 2 | 0.1 | 0.01 | 0.1 | |
| 13 | 2 | 0.25 | 0.05 | 0.05 | |
| 14 | 3 | 0.5 | 0.01 | 0.1 | |
| 15 | 3 | 0.5 | 0.1 | 0.01 | |
| 16 | 3 | 0.5 | 0.01 | 0.01 | |
| 17 | 3 | 0.25 | 0.01 | 0.05 | |
| 18 | 3 | 0.1 | 0.1 | 0.1 | |

### RESULTS AND DISCUSSION

### Hydrodynamics and CFD Analysis

Suitable governing equations are applied for hydrodynamics study in the bioreactor and TFF parts to calculate the filtrate volume with constant fluid velocity and suitable pressure gradient under steady state flow conditions. Equation (6) has been utilised to get the prediction of pressure drop in the microchannels. Selection of the characteristic length is an arbitrary choice and will not affect the final solution as illustrated in table 12 and table 13

TABLE 12

Fluid properties under different channel. All measurements were undertaken with a width (µm) of 1000, a height of (µm) of 500, a viscosity (cPa) of 3.5 and a density (g/cm³) of 1.06

| Inlet Flow Rate (µl/min) | Total Length (mm) | Pressure Drop (Pa) | Velocity (m/s) | Reynold’s No | Flow Time (min) |
|---|---|---|---|---|---|
| 3 | 1000 | 24.57 | 0.0001 | 0.0202 | 166.67 |
| 3 | 1200 | 29.48 | 0.0001 | 0.0202 | 200.00 |
| 3 | 1500 | 36.85 | 0.0001 | 0.0202 | 250.00 |
| 3 | 2000 | 49.14 | 0.0001 | 0.0202 | 333.33 |
| 3 | 2200 | 54.05 | 0.0001 | 0.0202 | 366.67 |
| 3 | 2500 | 61.43 | 0.0001 | 0.0202 | 416.67 |

TABLE 13

Fluid properties under different flow rate. All measurements were undertaken with a width (µm) of 1000, a height of (µm) of 500, a viscosity (cPa) of 3.5 and a density (g/cm³) of 1.06

| Inlet Flow Rate (µl/min) | Total Length (mm) | Pressure Drop (Pa) | Velocity (m/s) | Reynold’s No | Flow Time (min) |
|---|---|---|---|---|---|
| 4 | 1000 | 32.76 | 0.00013 | 0.0269 | 128.21 |
| 4 | 1200 | 39.31 | 0.00013 | 0.0269 | 153.85 |
| 4 | 1500 | 49.14 | 0.00013 | 0.0269 | 192.31 |
| 4 | 2000 | 58.96 | 0.00013 | 0.0269 | 230.77 |
| 4 | 2200 | 65.52 | 0.00013 | 0.0269 | 256.41 |
| 4 | 2500 | 81.90 | 0.00013 | 0.0269 | 320.51 |

Other pressure losses associated with the measured pressure drop are inlet and exit bend losses. Bend pressure losses can be obtained within different number of bends in table 14.

TABLE 14

Bend pressure loss percentage calculations with different number of bends

| No. of Bends | Flow Rate (µl/min) | Density (g/cm³) | Bend Pressure Loss (Pa) | % of Pressure Loss |
|---|---|---|---|---|
| 12 | 3 | 1.06 | 1.48 | 0.99% |
| 16 | 3 | 1.06 | 1.97 | 1.32% |
| 20 | 3 | 1.06 | 2.47 | 1.65% |
| 24 | 3 | 1.06 | 2.96 | 1.97% |
| 28 | 3 | 1.06 | 3.45 | 2.30% |
| 32 | 3 | 1.06 | 3.95 | 2.63% |
| 36 | 3 | 1.06 | 4.44 | 2.96% |
| 40 | 3 | 1.06 | 4.93 | 3.29% |

### Hydrodynamics Study in TFF Part

Cerman-Kozeny relationship is used to obtain the permeability constant to solve the fluid parameters through microporous channels under suitable boundary conditions. Here different particle diameters and pore sizes are applied because of the variations in input particle sizes in table 15.

TABLE 15

Permeability constant for different particle diameters and pore sizes

| Particle Diameter, dₚ (µm) | Pore Size, ε(µm) | Permeability, K (/µm³) | Darcy Number, Da |
|---|---|---|---|
| 0.020 | 0.060 | 5.4323E-10 | 2.122E-22 |
| 0.021 | 0.063 | 6.9776E-10 | 2.726E-22 |
| 0.022 | 0.066 | 8.8616E-10 | 3.462E-22 |
| 0.023 | 0.069 | 1.1139E-09 | 4.351E-22 |
| 0.024 | 0.072 | 1.3869E-09 | 5.418E-22 |
| 0.025 | 0.075 | 1.7120E-09 | 6.688E-22 |

The pressure drop for the geometry can be predicted from equation 10 The limitation of the equation is that it gives mathematical error if the units of measurements in microns. Pressure drop over length in Micro-porous Channel, as shown in table 16.

$$\frac{Δ P}{L} = \frac{μ Q s i n h \left(\frac{h}{\sqrt{K}}\right)}{K h \left[2 \frac{\sqrt{K}}{h} \left[- 1 + cosh \left(\frac{h}{\sqrt{K}}\right)\right] - s i n h \left(\frac{h}{\sqrt{K}}\right)\right]} \qquad \text{(10)}$$

Where,
µ= Dynamic Viscosity
Q= Flow Rate
h= height/depth of channel
K= Permeability

TABLE 16

Pressure loss over length in microporous channel for different flow rates

| Flow Rate, Q (µl/min) | µQ.sin h.(h/√ k) | Kh | 2√k/h(-1+cosh(h/√k)) | sinh(h/√k) | $K h \left[2 \frac{\sqrt{K}}{h} \left[- 1 + cosh \left(\frac{\text{h}}{\sqrt{K}}\right)\right] - sinh \left(\frac{\text{h}}{\sqrt{K}}\right)\right]$ | ΔP/L (KPa) |
|---|---|---|---|---|---|---|
| 3 | 1.943 | 0.00002048 | 3.15145083 | 0.185 | 6.07529E-05 | 31.97 |
| 4 | 2.590 | 0.00002048 | 3.15145083 | 0.185 | 6.07529E-05 | 42.63 |
| 5 | 3.238 | 0.00002048 | 3.15145083 | 0.185 | 6.07529E-05 | 53.28 |
| 8 | 5.180 | 0.00002048 | 3.15145083 | 0.185 | 6.07529E-05 | 85.26 |
| 10 | 6.475 | 0.00002048 | 3.15145083 | 0.185 | 6.07529E-05 | 106.58 |

The result that are obtained in this study is just a prediction which depends on the geometry, fluid conditions and boundary conditions. According to the membrane selection parameters in the Pall Corporation article, the filtrate volumes can be estimated which is given in table 17.

TABLE 17

Filtrate volume prediction for different filter length area

| Length (m) | Membrane Area, A (m²) | Filtrate Flux Rate, J (LMH) | Process Time, T (Hours) | Filtrate Volume V, (ml) |
|---|---|---|---|---|
| 0.4 | 0.00032 | 10 | 0.50 | 1.6 |
| 0.5 | 0.0004 | 10 | 2.00 | 8.0 |
| 0.6 | 0.00048 | 10 | 1.00 | 4.8 |
| 0.7 | 0.00056 | 10 | 1.50 | 8.4 |
| 0.8 | 0.00064 | 10 | 2.00 | 12.8 |
| 1.0 | 0.0008 | 10 | 2.00 | 16.0 |

### Computational Fluid Dynamics Analysis

### Velocity Profile in Bioreactor Channel

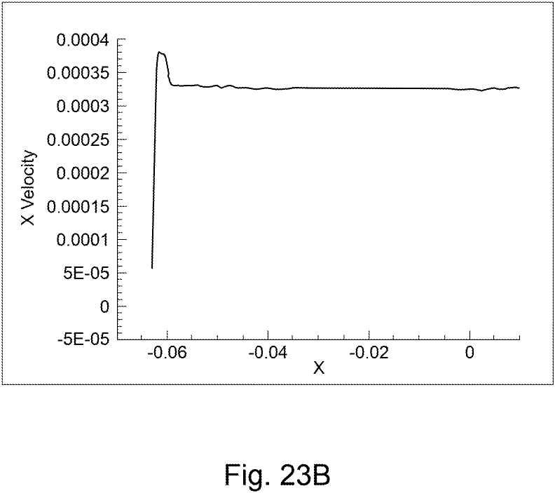

As FIGS. 23A and 23B demonstrates, the X direction velocity goes through the whole bioreactor channel. There are two different colours show the same velocity, which go between 0.0001 ms^-1 and 0.0002 ms^-1, the reason being that the yellow colour indexes positive direction and the green colour indexes negative direction. At the beginning, FIG. 23B shows that the velocity increased because the initial pump gives higher pressure and then declined to the constant velocity of about 0.00033 ms^-1 which is around two times higher than the predicted result.

### Tangential Flow Filtration Channel

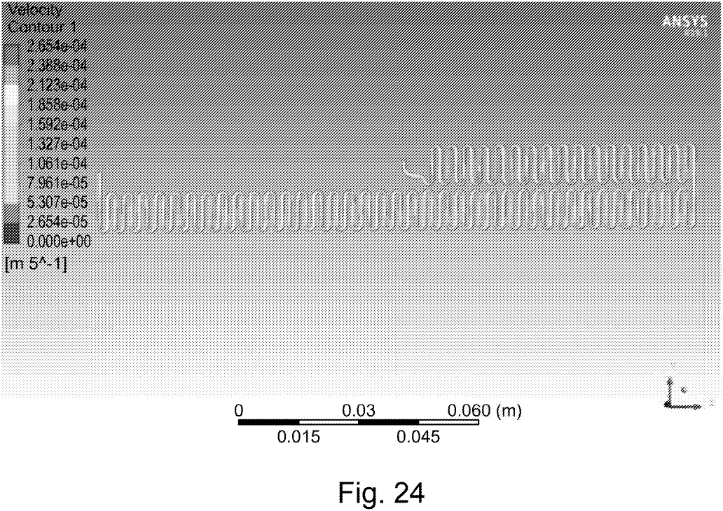

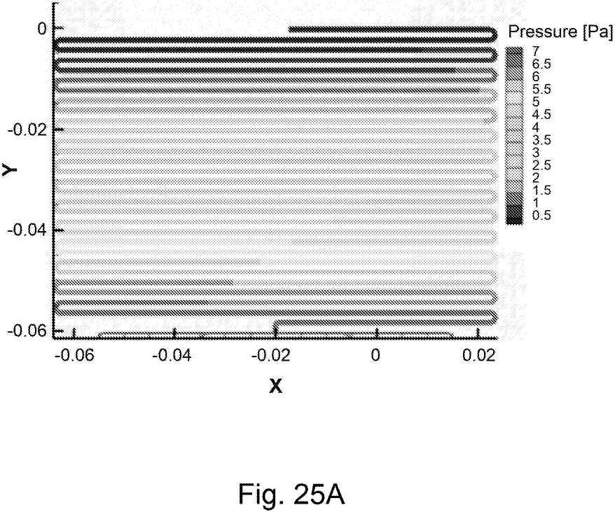

FIG. 24 shows the velocity in tangential flow filtration channel. The colours demonstrate that different velocity in microchannel are between 0.0001327 ms^-1 and 0.0001858 ms^-1 which is close to the inlet velocity. Moreover, the data from table 18 indicates that the outlet velocity is slightly declining from 0.000145 ms^-1 to 0.000138 ms^-1, as expected.

TABLE 18

Velocity magnitude
Velocity Magnitude (m/s)

| | |
|---|---|
| Inlet | 0.000145 |
| Outlet | 0.000138 |

### Pressure Contours in Bioreactors

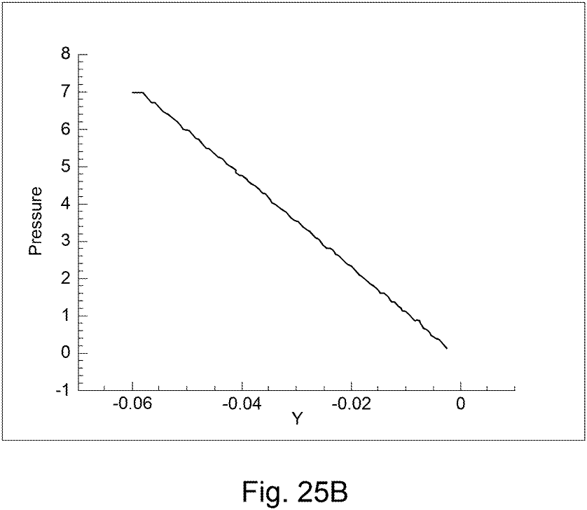

FIG. 25 illustrates the pressure drops in the bioreactor channel. The colours represent the different pressure values. FIG. 25B demonstrates that the pressure drops down as a consequence of having many bends in the bioreactor channel. Those different results have been considered in the design evolution.

### EXPERIMENTAL RESULTS

### PDMS Masks Tests

### Bioreactors

As different versions of moulds have been realised through the present work, the evolution of the result has been observed and will be explained.

### A) First Mould Generation

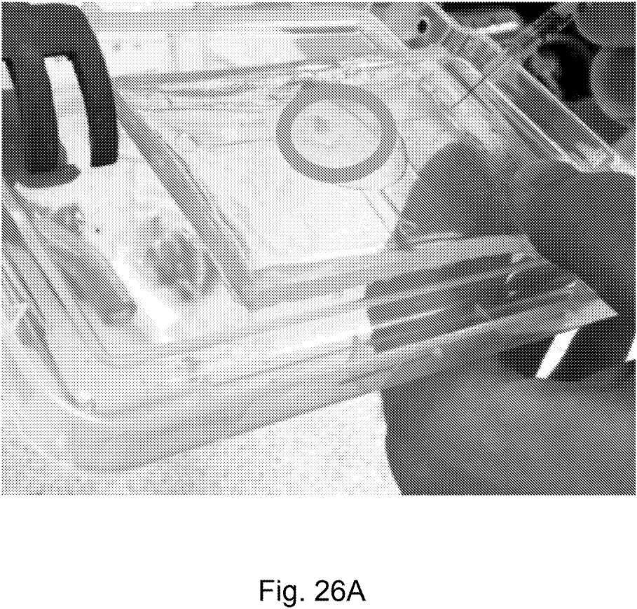

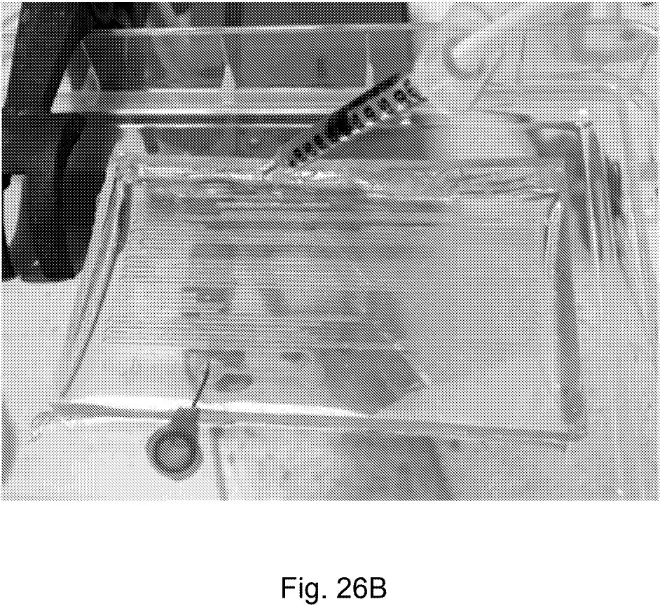

First, both first version of the two bioreactors have been tested. The different sealing options, Kapton tape and second PDMS layer have been experimented, and results have been extracted. Both reactors have been curing over two days at 35° C. The two reactors have been firstly sealed with another flat PDMS’ layer. Green water has been introduced in one of the inlets of each masks thanks to a syringe. FIG. 26A and FIG. 26B show the first result about the fluid behaviour for each mask. The two pictures show that on the batch bioreactor, when the green water is introduced in the mask, the water is contouring the middle of the reactor. This has been explained by the fact that the middle of the reactor has collapsed during the curing as it is possible to see on blue section of FIG. 26A. Furthermore, FIG. 26B shows that green water sample has gone through the T junction of the masks and followed the main channel pathway. However, the sample has avoided some areas. This can be explained by the size of the channel’s walls, that couldn’t support the pressure and collapse during the curing process.

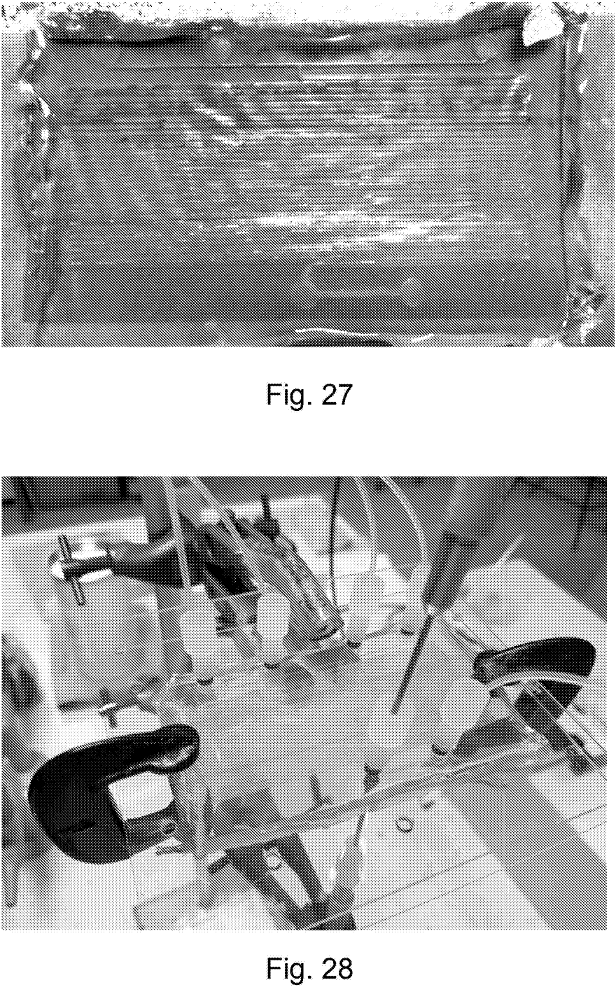

Secondly, the continuous flow reactor sealed with Kapton tape has been tested. FIG. 27 shows the fluid behaviour inside the new set up. The fluid (dark green) has followed the pathway correctly. The sealing has worked better than the previous one. However, due to the size of the channel the curing did not work properly again, and some parts of the mask did not peel properly from the mould creating some defects zone in the mask.

From all the observations made on the different tests, the design has been changed. In addition, it has been decided to create a platform where the PDMS mask can be placed between two acrylic plates, increasing the pressure between the two PDMS layers and, therefore, avoiding the channel to collapse when testing the masks. These two acrylic plates would also allow to connect fittings and tubing to the chip.

FIG. 28 shows that Kapton sealed masks have been placed between the two plates, and has been connected with tubing, fittings and some sensing instrument to allow first PH and absorbance testing and measurements.

### B) Second Mould Generation

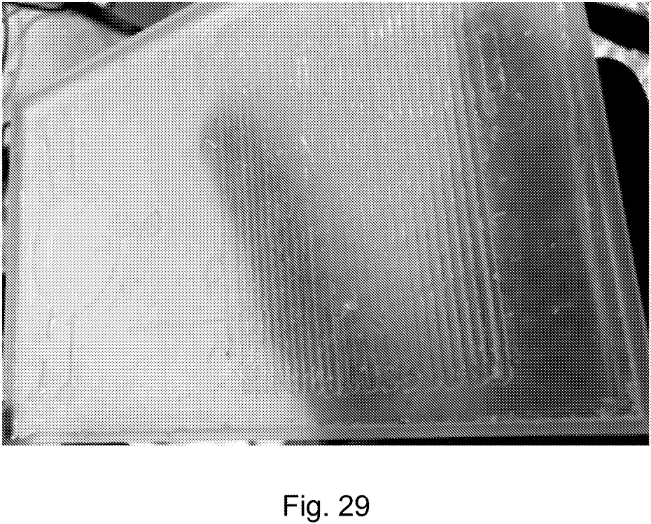

Using the high temperature resistant material to print the second mould generation the masks have been casted with a curing temperature of 70° C. for four hours. As FIG. 29 shows, the mould after use, the side in direct contact with the mould has bonded with it. Small parts of the PDMS mask could not be peeled off and removed from the mask. Specially between the channels, thin layers of PDMS were sticking on the mask. Although the curing has worked properly, the peeling of the mask from the mould has failed. After trying to clean the mould as much as possible with isopropanol, another tentative was given with a curing temperature of 100° C. for 35 minutes. The same problem occurred unfortunately. An explanation of such problem can be given by the fact the high temperature resistant material from the 3D printers has bonded with the PDMS layer directly in contact with it. The walls may have, as well, an impact on destabilizing the curing homogeneity of the whole mask.

### Vertical Flow Filtration Module

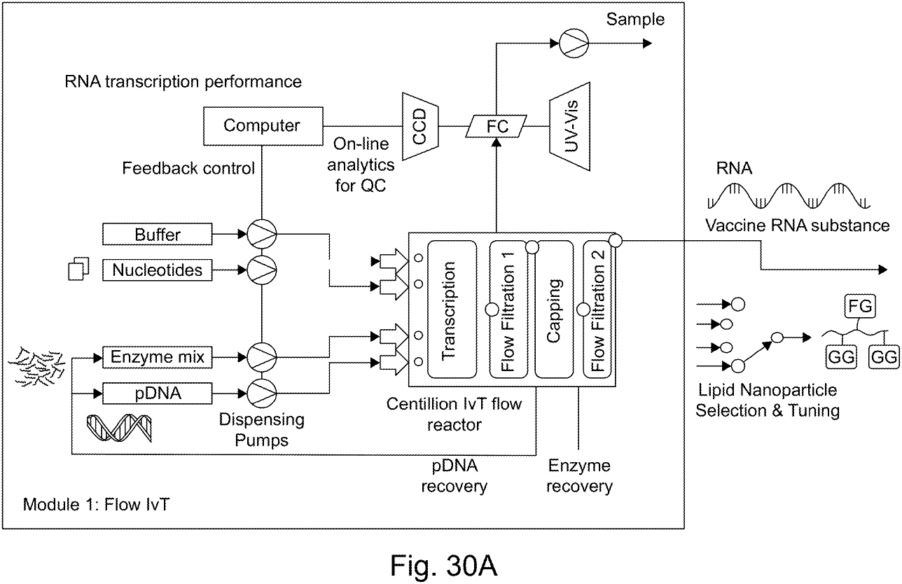

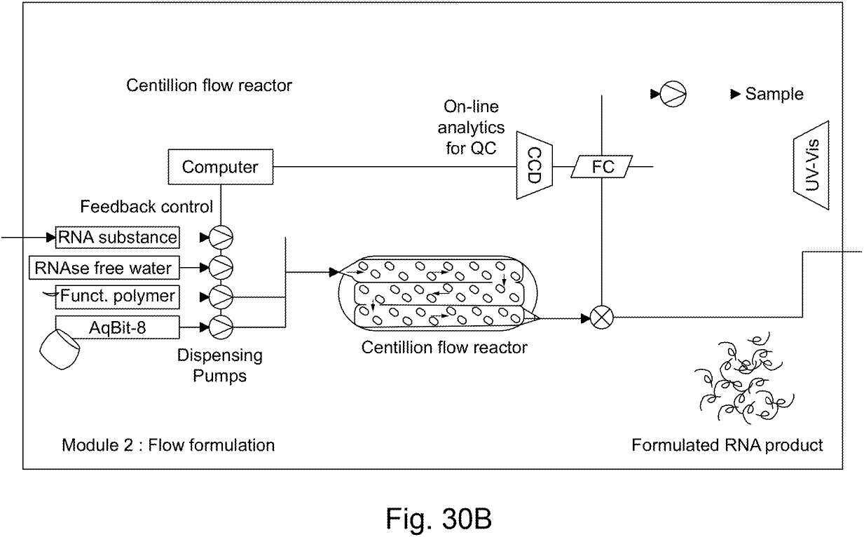

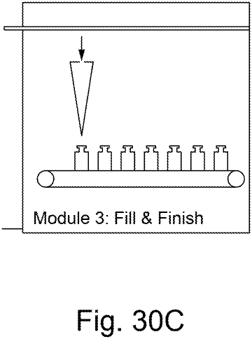

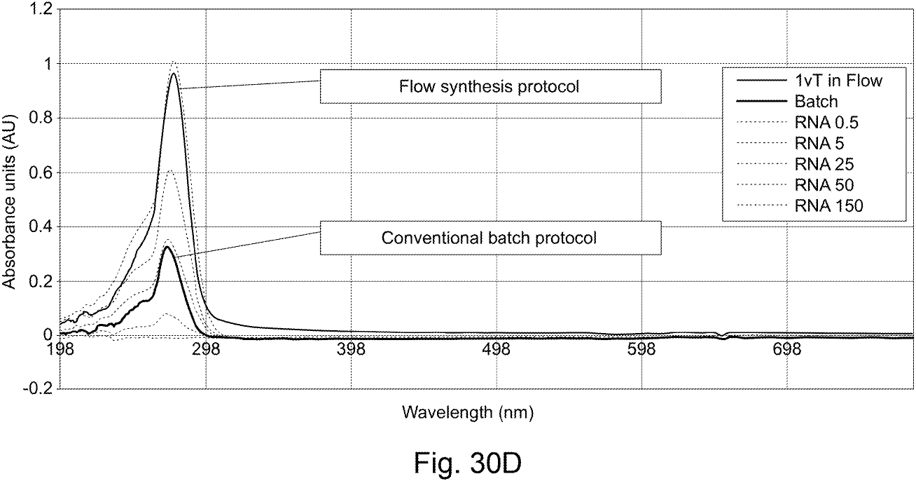

Only one version of the vertical TFF mould have been printed using the high temperature resistant material. As for the bioreactor masks the same process have been followed and a PDMS sample, poured in a foil paper recipient, has been placed in the oven with the two masks in order to compare the different aspect and result of the process. FIG. 30A and FIG. 30B show the aspect of the masks as well as the sample cured in the oven. As for the bioreactor mask, the PDMS in contact with the mould has bonded with it. When peeling it off from the mould, the mask has cracked and broken as shown in FIG. 30A. However, the sample cured in a foil paper has cured without any defaults and nothing happen when peeling it off from the foil. On top of that, the sample has casted perfectly the shapes of the foil as shown in FIG. 30B.

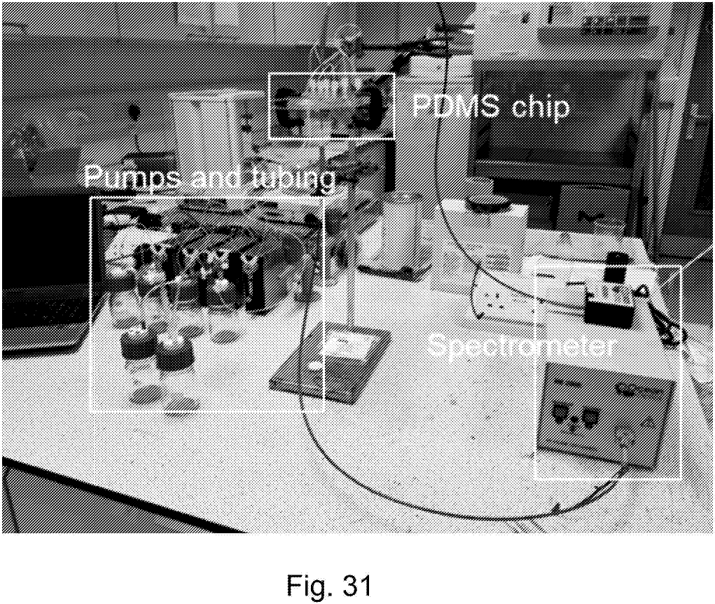

From these different observations some analysis can be made, and result found:
The chemistry and the protocol cannot be challenged as the sample in the foil worked.
The same defaults have occurred several times with the different masks. Therefore, the high temperature material has a negative impact on the PDMS mask casting and should not be used for such experiments
The first material used for the first experience should be kept and the curing parameters should be set at ambient temperature with 48 hours of curing, to let
the PDMS mask cast properly and avoid any heat deflection from the mould. Despite of these results, a new platform has been created, as shown in FIG. 31.

Based on the integrated experiment design, all the facilities have been collected in the lab, and the experimental platform has been set up to verify the effectiveness of the system. The built experimental platform in the lab is shown in FIG. 31, the PDMS chip is fixed and clamped by a fixture and connected the pumps and chip by tubing and fittings. Two optical fibres are installed vertically to each other: a source fibre that is connected to the light source, and a collection fibre connected to the mini spectrometer (Ocean Optics USB2000+).

### Absorbance Measurement and Digital Output

### Precise Absorbance Measurement

For precise absorbance measurements, the baseline needs to be set up. Firstly, record the spectrum of the background source of radiation without the sample. Secondly, repeat the experiment by adding the sample in the absorption path. Thirdly, subtract the first spectrum from the second one and get a clean absorption spectrum of that sample. To cancel the noise effect, the background spectrum should be subtracted from both sample and reference spectra. So, equation (9) should become:

$$\text{Absorbance} = - {\text{log}}_{10} \frac{{I}_{s a m p l e} - {I}_{B}}{{I}_{R e f} - {I}_{B}} \qquad \text{(11)}$$

Where: Iₛₐₘₚₗₑ is the light intensity after passing through the sample, I_{B} is the background light intensity, which is recorded by the spectrometer with the light source switched-off or blocked and without sample. I_{Ref} is the reference light intensity.

### UV-Vis Data Analysis

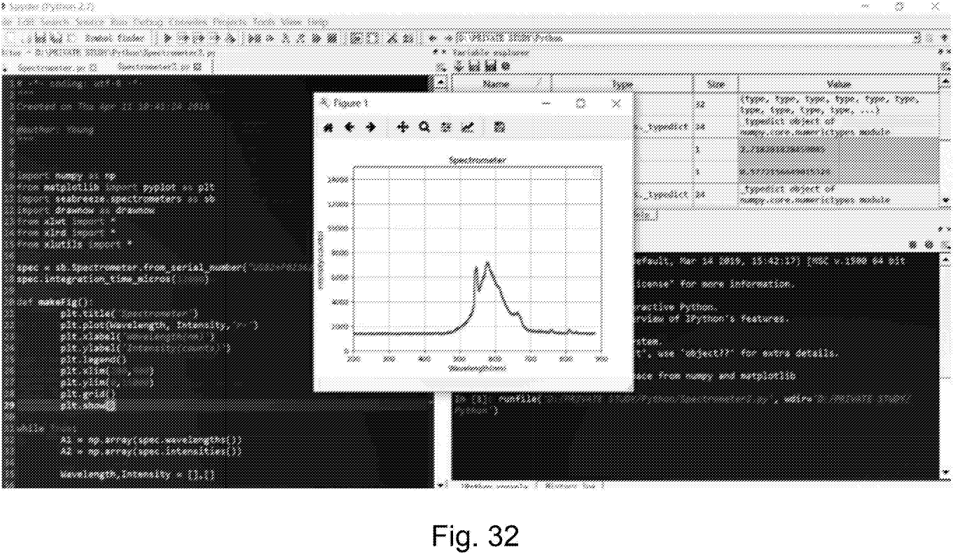

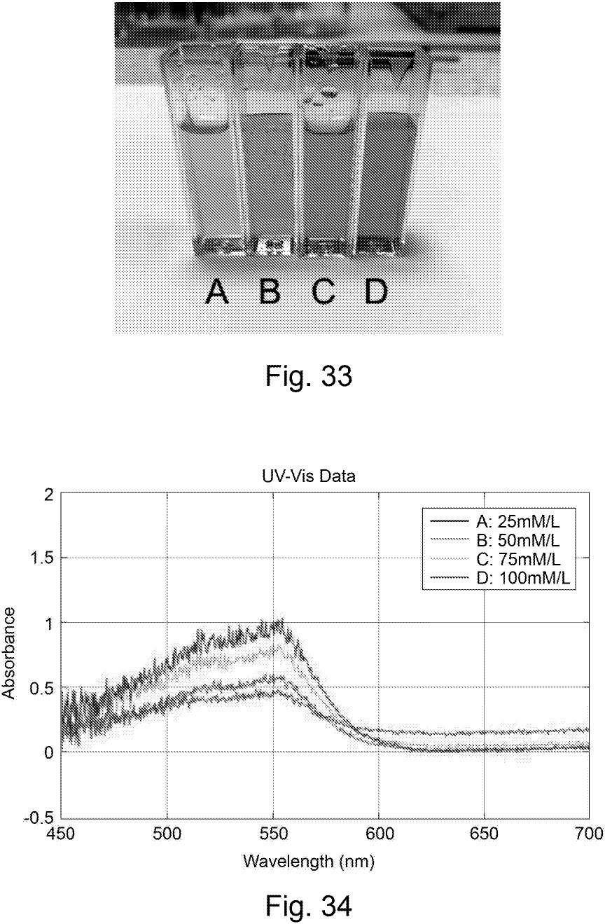

Through Python-Seabreeze API, it’s easy to get the real-time data from the UV-Vis spectrometer and plot the figure of intensities against wavelength. As FIG. 32 shows, the light intensity reaches a peak value when the wavelength is around 580 nm. To verify the relationship between absorbance and wavelength at different concentrations, various concentrations of Vitamin B12 solutions have been used, which are A (25 mM/L), B (50 mM/L), C (75 mM/L), D (100 mM/L) respectively, see FIG. 33. The UV-Vis spectral result is shown in FIG. 34, the absorbance increases with the increase of sample concentration, and the absorbance reaches the maximum at a wavelength of 550 nm.

### PH Sensor Calibration and Digital Output

### PH Sensor Calibration

To ensure accuracy, the PH probe used for the first time needs to be calibrated. two standard buffer solutions are used to calibrate the PH sensor, which are of 4.0 and 7.0. Following the calibration step provided by DFROBOT, the calibration has been completed.

### PH Value Plot in Real Time

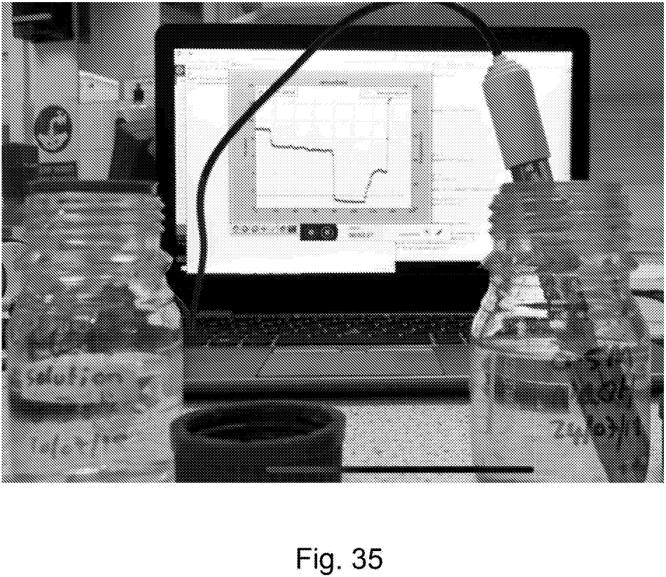

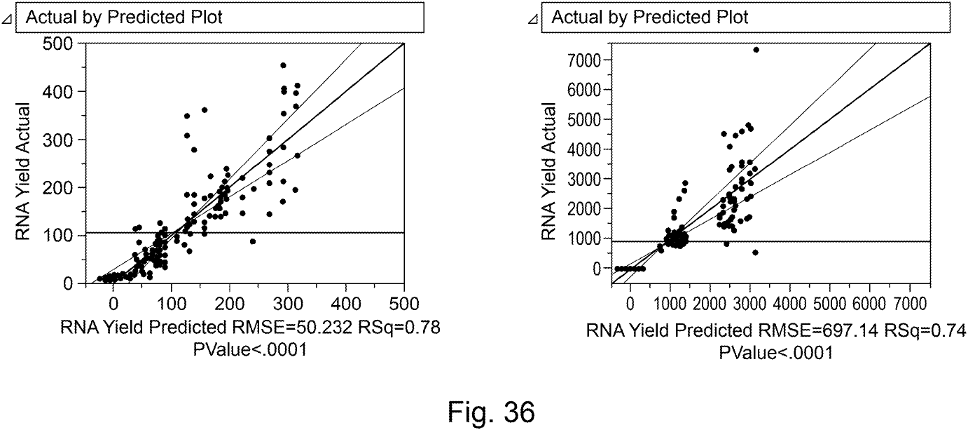

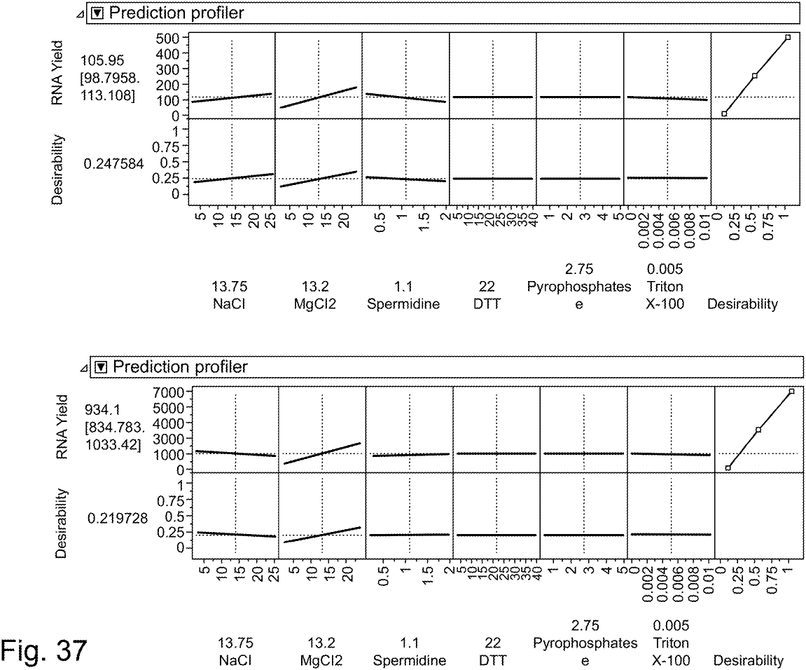

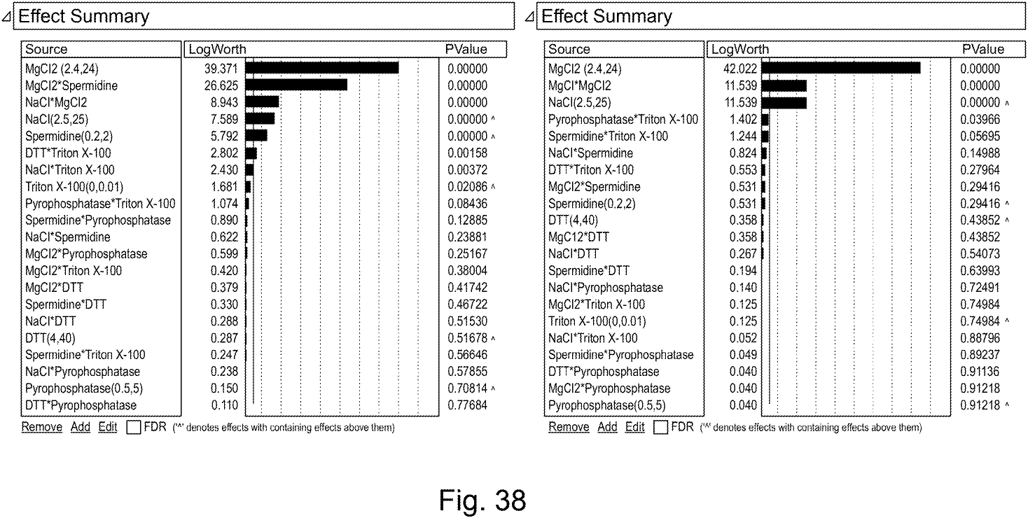

The next step consisted in testing the effectiveness of the PH sensor with acid and alkaline solutions. The result, in FIG. 35, shows that real-time figure dynamic changes follow PH changes, which proves its validity.

### Discussion

### Critical Result Evaluation

The work and results obtained are effective attempts of implementing lab-on-a-chip technology to achieve continuous flow reaction with maximum RNA output. Applying the SBCE methodology has allowed to build an entire design framework that has been followed from the beginning to the end of the project. The modular design of the device having divided functional subsystems enables inclusion of different innovative solutions for mixing, reaction and filtration and purification unit operations. FIGS. 30a to 30c show a schematic of the integrated system that includes a downstream module for formulation. In particular, FIGS. 30a to 30c is an illustration of an example embodiment of the present flow system integrated with a downstream formulation system using a modular flow reactor (module 2) (for example WO 2013/050764 and M. Jreissat, 2016, “A novel flow system for the concurrent product and process design of emulsion-based formulations”, Brunel University London); and integrated UV-Vis probe and attached to a conventional fill and finish vaccine production line.

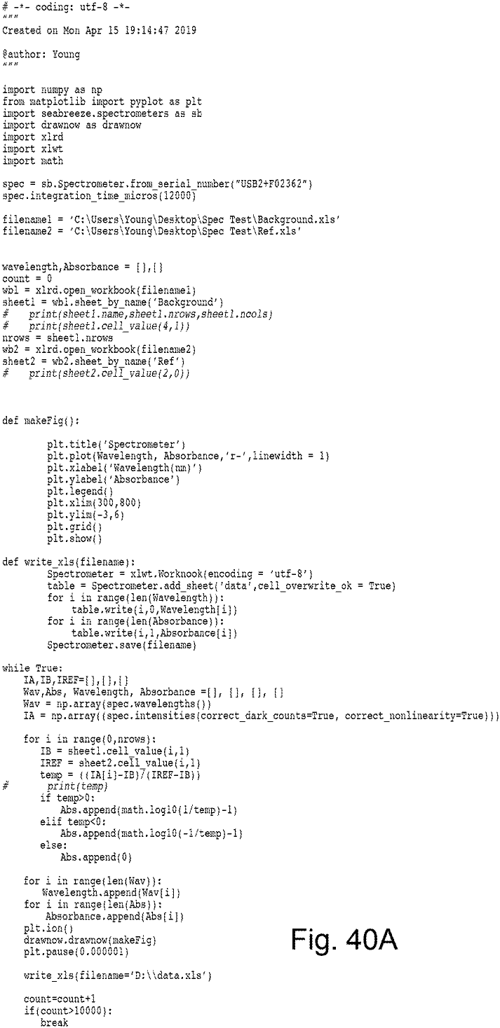

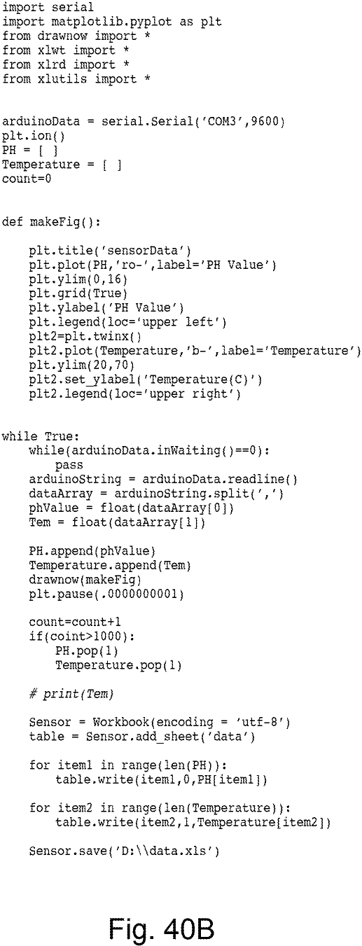

The synthesis performance of the device is shown in FIG. 30d being a graph of absorbance vs wavelength for an output solution of a flow reactor of the embodiment of FIGS. 30a to 30d. FIG. 40A is an illustration of a source code of absorbance data and real-time plot (Absorbance against Wavelength) in Python and FIG. 40B is an illustration of a source code of live plot PH data in Python.

The product is evaluated using UV-Vis spectroscopy against a conventional batch protocol graph of is the output solution of the flow reactor evaluated using UV-Vis spectroscopy against a conventional batch protocol. One can envisage the significantly higher productivity of the present method compared to the batch protocol by the higher peaks in the graph indicating higher levels of concentration of RNA. The UV-Vis spectra shown in FIG. 30d compare the RNA substance obtained using a conventional batch protocol and the continuous flow protocol of the present invention. The higher peak of the flow protocol compared to the batch corresponds to the higher concentration of the nucleic acid present in the solution. The dotted lines correspond to sample of known concentrations of RNA and are included to aid comparison between those obtained in batch and flow.

Two different design solutions have been developed for the reactor which allow to change the RNA manufacturing from a largely manual batch process (conventional RNA synthesis) to a continuous flow format. In fact, the continuous flow reactor of the present invention is innovative and proven to work. The present invention represents a step change in the prior art as it embodies a well-developed apparatus and process and demonstrating its operation and performance in tackling the rapid scale up and productive manufacture of RNA based substances in continuous flow format with automated computer control.

The filtration module has been deeply studied and analysed to meet the requirements of continuous flow processing and ability to integrate with a flow reactor while being easy to manufacture and scale using common manufacturing methods. Albeit tangential flow filtration, is a common technique that is proved to be effective in RNA purification processes (A. Eon-Duval et al., 2002), the present invention comprises a novel filtration device that has been purposely designed and built developed with the aim of being configured for use as a continuous flow system, be easy to manufacture, be capable of integration within a continuous flow system and allow scalable and cost-effective manufacture of the device. The present system may be integrated as a micro factory that can allow the automated and compliant manufacture of therapeutic nucleic acid based substances at the point of use. In addition, the modular design allows the recirculation of certain components such as the enzymes and the plasmid DNA with the use of this filtration system reducing significantly the overall cost of the substance produced.

Regarding the whole system, a new integrated platform has been developed in order to run tests and experiments. The system has been prototyped using acrylic pressure plates and that also comprise all the ports for the pumps, sensors and fittings for inlet and outlet. It allows to test a continuous flow reaction or filtration while getting instantaneous feedback from the different elements and stages of each process. Furthermore, the system has been prototyped in a modular way that allows an easy to use framework for running the experiments. 3D printing has been the chosen tool for manufacturing the moulds due to its high flexibility, low cost and short lead times. The construction of the modules is readily transferable to commercial manufacture using techniques such as micro machining on glass, metals such as stainless steel and poly methyl methacrylate PMMA (acrylic) substrates and also photolithography and deep reactive-ion etching on substrates including photosensitive glass and silicon. Furthermore, for large scale manufacture of the modules injection moulding can be used for PMMA and polycarbonate materials of construction.

The UV-Vis spectra demonstrate the ability of the present invention to provide reliable on-line and in-situ analysis of the reaction solution contents in real-time. This also offers a close feedback loop to direct fluid flow and regulate the temperature, pH and the dispensing of the reactants in the flow system to maintain process conditions throughout.

1. A method of RNA synthesis comprising:
introducing into a first fluid flow module, via a plurality of inlet ports, a plurality of reactants comprising: at least one nucleoside triphosphate (NTP), a reaction buffer, and DNA, a DNA based compound or a DNA based mixture;
allowing at least some of the reactants to react within a reaction channel or well within the first module of the flow system;
retaining or recirculating the DNA at the first reactor module and allowing reaction products of the reactants to flow into a first fluidic filtration module; and
filtering the reaction products within the first filtration module.

2. The method as claimed in claim 1 wherein the at least one nucleoside triphosphate (NTP) is a solution of at least one nucleoside triphosphate (NTP).

3. The method as claimed in claim 1 wherein the at least one nucleoside triphosphate (NTP) comprises any one or a combination of a adenosine triphosphate (ATP); cytidine triphosphate (CTP); guanosine triphosphate (GTP); uridine-triphosphate (UTP).

4. The method as claimed in claim 1 wherein the DNA is plasmid DNA.

5. The method as claimed in claim 1 the plurality of reactants further comprise any one or a combination of an enzyme mix, a salt solution, RNA polymerase.

6. The method as claimed in claim 1 comprising recirculating at least some of the plurality of reactants from an outlet of the first filtration module to an inlet region of the first reactor module.

7. The method as claimed in claim 1 comprising delivering at least some of the filtered reaction products from the first filtration module into a second fluid flow reactor module in combination with inputting a capping enzyme into the second reactor module.

8. The method as claimed in claim 7 comprising delivering the fluid output from the second reactor module to a second fluid flow filtration module.

9. The method as claimed in claim 8 comprising recirculating any unreacted NTP to an inlet region of the first reactor module and recirculating any unreacted capping enzyme to the inlet region of the second reactor module.

10. The method as claimed in claim 1 wherein the salt solution comprises MgCl₂.

11. The method as claimed in claim 1 wherein the RNA polymerase comprises T7 polymerase.

12. RNA or an RNA based compound prepared by the method of claim 1.

13. Use of RNA or an RNA based compound prepared by the method of claim 1 in the preparation of a vaccine.

14. Fluid flow filtration apparatus comprising:
a first elongate fluid flow channel having an inlet;
a second elongate fluid flow channel having an outlet;
a permeable membrane positioned to partition the first channel from the second channel along the respective lengths of the first and second channels such that a permeate may pass from the fluid within the first channel via the membrane into the second channel along the lengths of the first and second channels.

15. The apparatus as claimed in claim 14 wherein a majority of the length of the first channel is positioned adjacent a majority of the length of the second channel via the membrane.

16. The apparatus as claimed in claim 14 wherein a pore size of the membrane is in a range 100 to 1000 kDa; 100 to 800 kDa; 200 to 800 kDa or 200 to 600 kDa; 300 kDa to 10 MDa.

17. The apparatus as claimed in claim 14 comprising a first plate in which the first channel is formed and a second plate in which the second channel if formed, the membrane sandwiched between respective opposed faces of the first and second plates.

18. The apparatus as claimed in claim 14 wherein the first and second channels each comprise a series of straight sections and bent sections.

19. The apparatus as claimed in claim 18 wherein the first and second channels comprise respective serpentine profiles in their lengthwise directions.

20. The apparatus as claimed in claim 14 wherein the first and second channels are open along their lengths and positioned in direct contact with the membrane that, in part, defines a lengthwise wall or face of the first and second channels.

21. The apparatus as claimed in claim 14 wherein a pore size of the membrane is less than an average molecular size of an RNA molecule.

22. A fluid flow system for the processing of a fluid comprising:
a first reactor module having a reaction flow channel or well, at least one inlet and at least one outlet;
a first filtration module having a fluidic filtration region, at least one inlet provided in fluid communication with the outlet of the first reactor module and at least one outlet;
wherein the first filtration module comprises the fluid flow filtration apparatus as claimed in claim 13.

23. The system as claimed in claim 22 comprising a second reactor module having a reaction flow channel or well, at least one inlet and an outlet, said inlet provided in fluid communication with the first filtration module.

24. The system as claimed in claim 23 further comprising a second filtration module having a fluid filtration region, at least one inlet and outlet, said inlet provided in fluid communication with the outlet of the second reactor module.

25. The system as claimed in claim 22 comprising a fluid injection port provided in fluid communication with an inlet region of the first filtration module.

26. The system as claimed in claim 22 comprising a first recirculation conduit extending between a region of the outlet of the first reactor module and the at least one inlet of the first reactor module.

27. The system as claimed in claim 22 comprising a second recirculation conduit extending between a region of the outlet of the first filtration module and the outlet of the first reactor module.

28. The system as claimed in claim 27 comprising a third recirculation conduit extending between a region of the outlet of the second filtration module and the inlet of the first reaction module.

29. A method of filtering a fluid using fluid flow filtration apparatus comprising:
driving a fluid through a first elongate fluid flow channel from an inlet;
forcing a permeate component of the fluid through a membrane extending along the first channel and into a second elongate fluid flow channel; and
retaining a retentate component of the fluid within the first channel;
wherein the membrane is positioned to partition the first channel from the second channel along their respective lengths such that the permeate may pass from the first channel via the membrane into the second channel along the respective lengths of the first and second channels.

30. The method as claimed in claim 29 wherein the step of driving the fluid comprises pressurizing the fluid within the first channel.

31. A flow system comprising:
at least one reactor module having a reaction fluid flow channel or well, at least one fluid inlet and at least one fluid outlet;
at least one flow driver to drive a flow of a fluid through the channel or well;
a first sensor to measure any one or a combination of a pressure, a temperature or a pH of the fluid within the system;
a second sensor to measure any one or a combination of a pressure, a temperature, a pH of the fluid within the system;
a reaction status monitoring device to monitor a characteristic of a fluid within the system indicative of a status of reaction of at least two chemical components of the fluid within the system;
a control unit to receive data from the at least one or a combination of the first sensor, the second sensor and the reaction status monitoring device and to control at least one characteristic of the fluid within the system.

32. The system as claimed in claim 31 wherein the characteristic of the system is any one or a combination of:
a pressure of the fluid within the system;
a temperature of the fluid within the system;
a pH of the fluid within the system;
a volume or ratio of one or more chemical components of the fluid within the system;
a flow rate of the fluid within the system.

33. The system as claimed in claim 32 wherein the control unit comprises a CPU, a PCB, a PLC, a PC, a processor chip, a handheld electronic device.

34. The system as claimed in claim 33 wherein the additional sensors comprise any one or a combination of temperature, pH, pressure, flow rate, flow volume or spectroscopic sensors.

35. A method of processing a fluid using fluid flow apparatus comprising:
introducing at least one fluid into a reactor module via at least one inlet;
driving the fluid to flow through or within a reaction flow channel or well using at least one flow driver and outputting the fluid at an outlet;
measuring at least one or a combination of pressure, temperature or pH of the fluid within the fluid flow apparatus;
monitoring a reaction status of chemical components within the fluid within the fluid flow apparatus; and
controlling at least one characteristic of the fluid within the fluid flow apparatus using a control unit in response to at least one or a combination of the measuring of the pressure, pH, temperature and/or reaction status of the chemical components of the fluid.

36. The system as claimed in claim 35 comprising a plurality of reactor modules and filtration modules coupled together in fluid communication.

37. The system as claimed in claim 35 wherein the flow driver is at least one pump and optionally a syringe pump.

38. The system as claimed in claim 35 wherein the fluid analysis sensor comprises a UV-Vis spectroscope for absorbance or fluorescence analysis in the range 190 nm to 1000 nm.
